(19) United States
(12) Patent Application Publication
(10) Pub. No.: US 2005/0277611 A1
(43) Pub. Date: Dec. 15, 2005

(54) Cationic cardiolipin analoges and its use thereof

(75) Inventors: Moghis U. Ahmad, Wadsworth, IL (US); Krishnudu Kasireddy, Vernon Hills, IL (US); Imran Ahmad, Wadsworth, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Neopharm, Inc., Lake Forrest, IL (US)

(21) Appl. No.: 11/106,406

(22) Filed: Apr. 14, 2005

Related U.S. Application Data
(63) Continuation of application No. PCT/US03/33099, filed on Oct. 16, 2003.
(60) Provisional application No. 60/419,277, filed on Oct. 16, 2002.

(51) Int. Cl. A61K 48/00; A61K 38/17; A61K 9/127

(52) U.S. Cl. 514/44; 424/450; 530/352; 554/78; 536/18.6

(57) ABSTRACT

The invention provides cationic cardiolipin compounds, and methods for synthesizing and using them in liposomal formulation, gene transfection, etc. In particular, the invention provides liposomes comprising cationic cardiolipin analog, pharmaceutical compositions comprising cationic cardiolipin analogs, and methods of using such liposomes and compositions, in delivering active pharmaceutical agents to treat human and animal diseases and/or in diagnostic assays.

### CROSS REFERENCE TO RELATED PATENT APPLICATIONS

This application is a continuation of PCT/US03/33099 filed on Oct. 16, 2003, which claims priority to U.S. Provisional Application No. 60/419,277 filed on Oct. 16, 2002. The disclosures of these applications are incorporated herein in their entireties by reference thereto.

### FIELD OF THE INVENTION

This invention pertains to cationic cardiolipin molecules, their methods of preparation and use, and compositions comprising cationic cardiolipin.

### BACKGROUND OF THE INVENTION

A need for synthetic phospholipids has developed, in part, from their use in liposomes, which have become useful carriers of active therapeutic agents, enzymes, antibiotics, antigens, and hormones, among other compounds (Tyrell, et al. 1976). Cationic liposomes are recognized as an important means to assist with the delivery of anionic species such as genes or other nucleic acids to cells (Miller, 1998). Cationic liposomes are thought to interact electrostatically with negatively charged nucleic acid sequences to form complexes that facilitate penetration of these agents into cells. Thus, cationic lipids could play a role in delivering anionic agents into target cells and organs of patients in the treatment of disease. Consequently, a need has arisen for the development of new synthetic methods for structurally well-defined lipids. (Bhattacharya et al. 1999).

The present invention relates to cationic cardiolipins which are used to enhance delivery of biologically active agents, particularly polynucleotides, proteins, peptides, and drug molecules, by facilitating transmembrane transport or by encouraging adhesion to biological surfaces. It relates particularly to cationic cardiolipins comprising ammonium groups. Some bioactive substances do not need to enter cells to exert their biological effect, because they operate either by acting on cell surfaces through cell surface receptors. However, many natural biological molecules and their analogs, including proteins and polynucleotides, or foreign substances, such as drugs, which are capable of influencing cell function at the subcellular or molecular level are preferably incorporated within the cell in order to produce their effect. For these agents, the cell membrane presents an impermeable selective barrier.

A major advance in the area of DNA transfection was the discovery that certain synthetic cationic lipids, such as N-[1-(2,3-dioleyloxy)propyl]-N,N,N-trimethylammonium chloride (DOTMA), in the form of liposomes or small vesicles, could interact spontaneously with DNA to form of lipid-DNA complexes that are capable of fusing with the negatively charged lipids of the cell membranes, resulting in both uptake and expression of the DNA (Felgner, et al. 1987). The well-known Lipofectin™ reagent (Bethesda Research Laboratories, Gaithersburg, Md.), an effective agent for the delivery of highly anionic polynucleotides into living tissue culture cells, comprises positively charged polynucleotides to form complexes. In part, the effectiveness of cationic lipids as cytofectins is thought to result from their enhanced affinity for cells, many of which bear regions of high negative charge on their membrane surfaces. Also the presence of positive charges on a lipid aggregate comprising a cationic lipid enables the aggregate to bind polyanions, especially nucleic acids. Lipid aggregates prepared in this way can spontaneously interact with negative charges on cell surfaces, fuse with the plasma membrane, and can efficiently deliver functional polynucleotides into cells.

Cardiolipin (also known as diphosphatidyl glycerol) constitutes a class of complex anionic phospholipids that is typically purified from cell membranes of tissues associated with high metabolic activity, including the mitochondria of heart and skeletal muscles (Grunner et al. 1985). However, known chromatographic purification techniques cannot resolve cardiolipin into discrete molecular species. As a result, the use of this component in drug formulations has been limited because the resulting formulations are not homogeneous. In addition, the compound is anionic which limits its use in cationic liposomes. The charge repulsion between cardiolipin and anionic agents can interfere with its use in many instances.

A cationic form of cardiolipin would attract anionic agents and would be more useful in drug delivery. Such a complex could also be used to stabilize hyrophobic compound in micelles and liposomes. New synthetic methods are needed that can be used to synthesize cationic cardiolipin. Synthetic methods for making cationic cardiolipin could be used to prepare homogeneous preparations of the compound.

Novel synthetic methods are needed that can be used to prepare large quantities of saturated and unsaturated cationic cardiolipin species having varying fatty acid chain lengths. Such methods would increase the availability of a wider variety of cationic cardiolipin species and would diversify the lipids available for development of new liposomal formulations containing active agents, which will have more defined compositions than those currently available.

The invention provides such methods and compositions. These and other advantages of the invention, as well as additional inventive features, will be evident from the description of the invention provided herein.

### SUMMARY OF THE INVENTION

The invention provides cationic cardiolipin compounds, and methods for synthesizing and using them. In particular, the invention provides liposomes comprising cationic cardiolipin analogs, pharmaceutical compositions comprising cationic cardiolipin analogs, and methods of using such liposomes and compositions, such as delivering active pharmaceutical agents to patients.

The cationic cardiolipin of the present invention can be incorporated into liposomes or other lipid formulations, which can also include active agents such as hydrophobic or hydrophilic drugs, nucleic acids such as antisense oligonucleotides or diagnostic agents. Such liposomes can be used to treat diseases or in diagnostic and/or analytical assays. The cationic cardiolipin is capable of facilitating transport of biologically active agents into cells. The cationic cardiolipin compounds of the present invention can be processed to form lipid aggregates together with bioactive agents and, as such, can be used as cytofectins.

### BRIEF DESCRIPTION OF THE DRAWINGS

FIG. 1 shows synthesis of the cationic cardiolipin analogs containing ether linked alkyl side chains.

FIGS. 2, 3, and 4 shows synthesis of the spacer cationic cardiolipin analogs containing ether linked alkyl side chains.

FIG. 5 shows synthesis of the spacer cationic cardiolipin analogs containing ether linked alkyl side chains.

FIG. 6 shows synthesis of the spacer cationic cardiolipin variant, cationic cardiolipin analogs containing ether linked alkyl side chains.

FIG. 7 shows synthesis of the cationic cardiolipin variant analogs containing ether linked alkyl side chains.

FIG. 8 shows synthesis of the spacer cationic cardiolipin analogs containing ester linked alkyl side chains.

### DETAILED DESCRIPTION OF THE INVENTION

The present invention provides cationic cardiolipin variants and analogs that include optically pure and/or diasteroisomers as well as methods for their synthesis and use. In one embodiment, the invention provides cationic cardiolipin variants and analogs having the general formula I or X and methods of their synthesis:

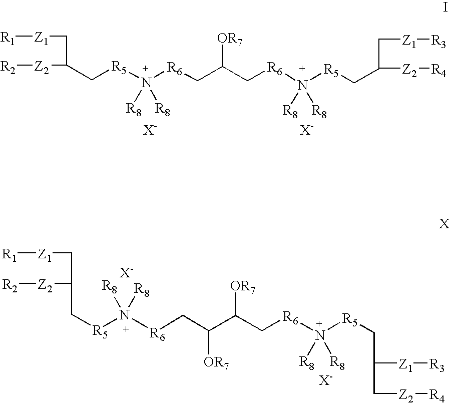

In Formulas I and X, Z₁ and Z₂ can be the same or different and are —O—C(O)—, —O—, —S—, —NH—C(O)— or the like. Also, in Formulas I and X, R₁, R₂, R₃, and R₄ can be the same or different and can be, independently, H, saturated or unsaturated alkyl, alkenyl, or alkynyl groups (typically, but not necessarily, C₁ to C₃₂), which can be optionally hydroxylated, aminenated, thiolated, epoxylated, cyclolated, PEGylated, halogenated, or substituted with combinations thereof. In Formulas I and X, R₅ and R₆ can be the same or different and can be, independently, either absent or comprise a linker comprising alkyl, substituted alkyl, cycloalkyl, substituted cycloalkyl (typically C₁ to C₃₂), or an alkyloxy group such as a PEGylated ether containing from 1 to 500 PEG (polyethylene glycol) units.

R₇ in Formulas I and X can be hydrogen, alkyl, substituted alkyl, alkyloxy, substituted alkyloxy, cycloalkyl, substituted cycloalkyl, alkenyl, alkynyl, alkanoyl, alkenoyl, alkynoyl, which can optionally be hydroxylated, aminenated, thiolated, epoxylated, cyclolated, PEGylated, halogenated, or substituted with combinations thereof; or an alkyloxy or substituted alkyloxy group such as PEGylated ether containing from 1 to 500 PEG (polyethylene glycol) units; an amino acid, a peptide, a peptidomimetic moiety, a dipeptide, a polypeptide, a protein, a carbohydrate, a saccharide or polysaccharide, a polyamine, a heterocyclic, a nucleoside, or a polynucleotide, or other like moiety. In formula X, the two R₇ substituents can be the same or different and can independently comprise such moieties. In Formulas I and X, the R₈ groups can be the same or different and can independently include a C₁ to C₂₅ saturated or unsaturated alkyl group, alkyloxy group, substituted alkyl group, or substituted alkoxy group. “X” in Formulas I and X is a non-toxic anion, such as chloride, bromide, iodide, and the like.

The term “alkyl” encompasses saturated or unsaturated straight-chain and branched-chain hydrocarbon moieties. The term “substituted alkyl” or “substituted alkoxy” and the like includes alkyl or alkoxy groups further bearing one or more substituents selected from hydroxy, alkoxy(of a lower alkyl group), mercapto (of a lower alkyl group), cycloalkyl, substituted cycloalkyl, halogen, cyano, nitro, amino, amido, imino, thio, —C(O)H, acyl, oxyacyl, carboxyl, and the like. The term “sugars” refers to any naturally occurring or unnatural sugars like glucose, mannose, allose, ribose, fucose, arabinose, galactose, 2-deoxy sugars, 3-deoxy sugars, 4-deoxy sugars, disaccharide and polysaccharides. The term “amino acid” refers to any naturally occurring or unnatural amino acid. This definition is intended to embrace substituted α-amino acids as well as non-α-amino acids. An α-amino acid is defined as an amino acid in which the amino group is attached to a carbon atom that is adjacent to the carboxylic acid group. The term “nucleotide sequence” refers to any one or more polynucleotides or polynucleotide segments or constructs (e.g., DNA or RNA oligomers, mRNA or pDNA). The nucleotide sequence may be provided in linear, circular (e.g., plasmid), or branched form as well as double-stranded or single-stranded form. The nucleotide sequences may comprise a conventional phosphodiester bond or a non-conventional bond (e.g., an amide bond, such as found in peptide nucleic acids (PNA)). The term “peptide” refers to a molecule comprising two or more amino acids that are linked by means of peptide bonds. A “peptide bond” or “peptide linkage” is a covalent bond formed by splitting out a water molecule between the carboxyl group of one amino acid and the amino group of a second amino acid, and has the chemical structure —C(O)—NR—, where R is H, C₁ to C₁₅ alkyl. The term “polypeptide” refers to a polymer of more than nine amino acids that are linked by means of peptide bonds. The term “protein” refers to a high molecular weight polypeptide of amino acids, and includes, but is not limited to hormones, antibodies and certain antigens (Wheeler, Carl, J. patent no WO 00/73263 A1).

In a preferred embodiment, R₁, R₂, R₃, and R₄ in Formulas I and X are the same or different and are H, or C₁ to C₃₂ alkyl, alkenyl, alkynyl, which are optionally hydroxylated, aminenated, thiolated, epoxylated, cyclolated, PEGylated, or halogenated. Also, in a preferred embodiment, R₅ and R₆ in Formulas I and X are the same or different, and are a linker comprising C₁ to C₃₂ alkyl or an alkyloxy such as PEGylated ether of containing from 1 to 500 PEG units. Also, in a preferred embodiment, an R₇ substituent in Formulas I and X is hydrogen, alkyl, substituted alkyl, cycloalkyl, substituted cycloalkyl, a peptide, dipeptide, polypeptide, protein, carbohydrate, polysaccharide, heterocyclic, nucleoside, or polynucleotide.

In other preferred embodiments, at least one of R₅, R₆, R₇ and R₈ in Formulas I and X comprises an optionally substituted alkyl or optionally substituted alkyloxy group. For example, at least one of R₅ or R₆ (or both) in Formulas I and X can comprise an optionally substituted polyalkyloxy group containing from 1 to 500 alkyloxy groups, such as from 1 to 100 alkyloxy groups. Indeed, in a preferred embodiment, at least one of R₇ and R₈ in Formulas I and X comprises an optionally substituted alkyl, and R₅ and R₆ comprises an optionally substituted polyalkyloxy group. In an especially preferred embodment, at least one of R₅ and/or R₆ (or all of the R₅ and/or R₆ substitutents) is ethoxy (mono PEG) in Formulas I and X.

In another preferred embodiment, an R₇ substituent in Formulas I and X is an amino acid, folic acid, saccharide, peptide, polysaccharide, polypeptide, protein, polyamine, or peptidomimetic moiety. For example, a preferred R₇ substituent in Formulas I and X is histone, spermine, spermidine, or a derivative thereof. Another preferred R₇ substituent in Formulas I and X is a saccharide attached as a O-glycoside or C-glycoside, such as glucose, mannose, galactose, ribose, arabinose, allose, fucose, a 2-deoxy sugar, or the like. In yet another preferred embodiment, an R₇ substituent in Formulas I and X can include an L- or D-alpha amino acid having a positively charged group on the side chain, such as, for example, arginine, histidine, lysine, ornithine or analogs thereof. Also, in a preferred embodiment, an R₇ substituent in Formulas I and X comprises an amino acid, saccharide, peptide, polysaccharide, polypeptide, protein, polyamine, or peptidomimetic moiety having one or more positive charge. With respect to Formula X, which has two R₇ substituents, they can be the same or different and can independently comprise such groups.

In a preferred embodiment, the R₈ groups in Formulas I and X are CH₃. In another preferred embodiment, at least one of R₅, R₆, R₇ and R₈ in Formulas I and X comprises a substituted alkyl or substituted alkyloxy group. In the most preferred embodiment Z₁ and Z₂ in Formulas I and X are —O—C(O)— or —O—. R₁, R₂, R₃ and R₄ are the same and are a C₁ to C₃₂ saturated and/or unsaturated alkyl group, preferably between 10 and 24 carbon atoms. “X” in Formulas I and X is most preferably chloride or bromide ion.

In a preferred compound according to general formula I, R₅ and R₆ are absent; R₇ is hydrogen; R₈ is CH₃; X is bromide; Z₁ and Z₂ are oxygen; which is a cationic cardiolipin ether is having the structure II: 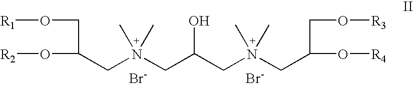
wherein, R₁, R₂, R₃ and R₄ are the same or different and are H, C₁ to C₃₂ alkyl, alkenyl, alkynyl groups;

In another preferred compound according to general formula I, R₅ and R₆ are absent; R₇ is hydrogen; R₈ is CH₃; X is bromide; Z₁ and Z₂ are —O—C(O)—; which is a cationic cardiolipin ester having the structure III: 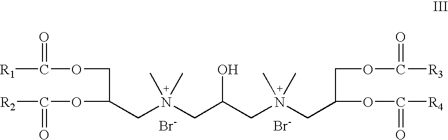
wherein, R₁, R₂, R₃ and R₄ are the same or different and are H, C₁ to C₃₁ alkyl, alkenyl, alkynyl groups.

In another preferred compound according to general formula I, R₅ is absent; R₆ is OCH₂CH₂; R₇ is hydrogen; R₈ is CH₃, X is bromide; Z₁ and Z₂ are oxygen; which is a cationic cardiolipin ether analog having the structure IV: 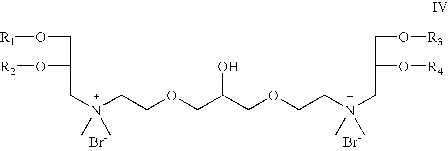
wherein, R₁, R₂, R₃ and R₄ are the same or different and are H, C₁ to C₃₂ alkyl, alkenyl, alkynyl groups.

In another preferred compound according to general formula I, R₅ is absent; R₆ is OCH₂CH₂; R₇ is hydrogen; R₈ is CH₃, X is bromide; Z₁ and Z₂ are —O—C(O)—; which is a cationic cardiolipin ester analog having the structure V: 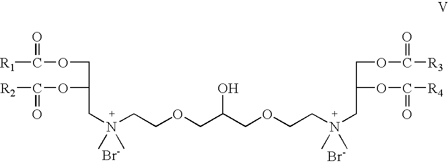
wherein, R₁, R₂, R₃ and R₄ are the same or different and are H, C₁ to C₃₁ alkyl, alkenyl, alkynyl groups.

In another preferred compound according to general formula I, R₅ is OCH₂CH₂; R₆ is absent; R₇ is hydrogen; R₈ is CH₃, X is bromide; Z₁ and Z₂ are oxygen; which is a cationic cardiolipin ether analog having the structure VI: 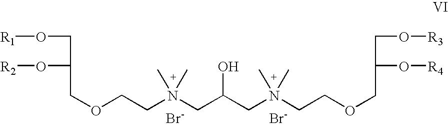
wherein, R₁, R₂, R₃ and R₄ are the same or different and are H, C₁ to C₃₂ alkyl, alkenyl, alkynyl groups.

In another preferred compound according to general formula I, R₅ is OCH₂CH₂; R₆ is absent; R₇ is hydrogen; R₈ is CH₃, X is bromide; Z₁ and Z₂ are —O—C(O)—; which is a cationic cardiolipin ester analog having the structure VII: 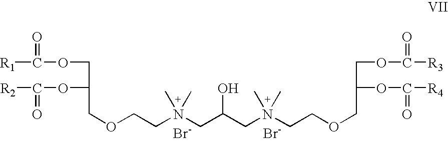
wherein, R₁, R₂, R₃ and R₄ are the same or different and are H, C₁ to C₃, alkyl, alkenyl, alkynyl groups.

In another preferred compound according to general formula I, both R₅ and R₆ are OCH₂CH₂; R₇ is hydrogen; R₈ is CH₃, X is bromide; Z₁ and Z₂ are oxygen; which is a cationic cardiolipin ether having the structure VIII: 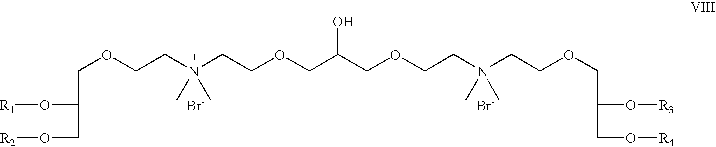
wherein, R₁, R₂, R₃ and R₄ are the same or different and are H, C₁ to C₃₂ alkyl, alkenyl, alkynyl groups.

In another preferred compound according to general formula I, both. R₅ and R₆ are OCH₂CH₂; R₇ is hydrogen; R₈ is CH₃, X is bromide; Z₁ and Z₂ are —O—C(O)—; which is a cationic cardiolipin ester having the structure IX: 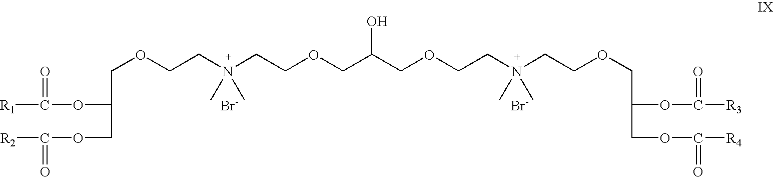
wherein, R₁, R₂, R₃ and R₄ are the same or different and are H, C₁ to C₃₁, alkyl, alkenyl, alkynyl groups.

In one preferred compound according to general formula X, R₅ and R₆ are absent; R₇ is hydrogen; X is bromide; R₈ is CH₃, Z₁ and Z₂ are oxygen; which is a cationic cardiolipin variant ether having the structure XI: 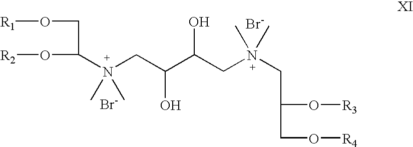
wherein, R₁, R₂, R₃ and R₄ are the same or different and are H, C₁ to C₃₂ alkyl, alkenyl, alkynyl groups.

In another preferred compound according to general formula X, R₅ and R₆ are absent; R₇ is hydrogen; X is bromide; R₈ is CH₃, Z₁ and Z₂ are —O—C(O)—; which is a cationic cardiolipin variant ester having the structure XII: 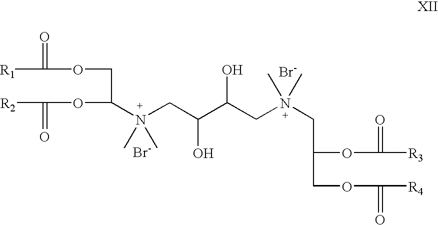
wherein, R₁, R₂, R₃ and R₄ are the same or different and are H, C₁ to C₃₁ alkyl, alkenyl, alkynyl groups.

In another embodiment, the invention provides cationic cardiolipin having the general structure XIII: 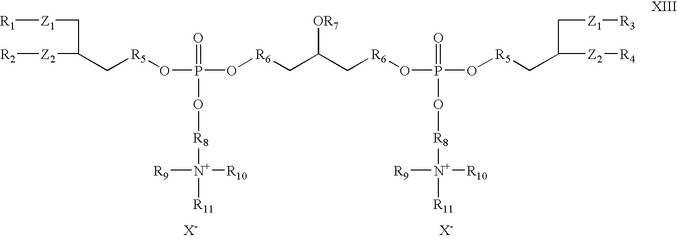
In compounds of Formula XIII, R₁, R₂, R₃, R₄, R₅, and R₆, X, Z₁, and Z₂ can be as described above with reference to Formulas I and X. Moreover, in Formula XIII, R₇, R₉, R₁₀, and R₁₁ can be as described above with reference to R₇ in Formulas I and X. In Formula XIII, R₈ is an alkyl, substituted alkyl, alkenyl, substituted alkenyl, alkyloxy or substituted alkyloxy, preferably C₂-C₃₂.

In one preferred compound according to general formula XIII, R₅ and R₆ are absent, R₈ is propyl; R₇, R₉, R₁₀, and R₁₁ are hydrogen; Z₁ and Z₂ are oxygen; X is chloride; which is a cationic cardiolipin variant ether is having the structure XIV: 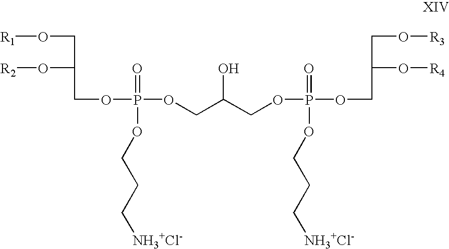
wherein, R₁, R₂, R₃ and R₄ are the same or different and are H, C₁ to C₃₂ alkyl, alkenyl, alkynyl groups.

In another preferred compound according to general formula XIII, R₅ and R₆ are absent, R₈ is propyl; R₇, R₉, R₁₀, and R₁₁ are hydrogen; Z₁ and Z₂ are —O—C(O)—; X is chloride; which is a cationic cardiolipin variant ester is having the structure XV: 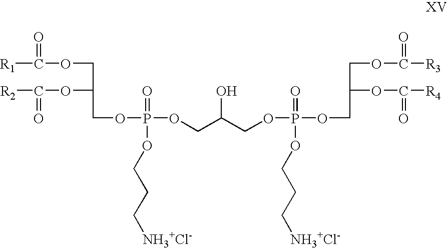
wherein, R₁, R₂, R₃ and R₄ are the same or different and are H, C₁ to C₃₁, alkyl, alkenyl, alkynyl groups.

Cationic cardiolipin molecules according to the present invention can comprise fatty acid/alkyl chains (e.g., at R₁, R₂, R₃, and R₄) of varying length and saturation/unsaturation. In general, the length of the fatty acid hydrocarbon chain ranges from about 2 to about 32 carbon atoms; however, the carbon chain is more typically between about 10 and about 24 carbon atoms (such as between 14 and 20 carbon atoms). Fatty acids typically are classified by the number of double and/or triple bonds in the hydrocarbon chain (i.e., unsaturation). A saturated fatty acid does not contain any double or triple bonds, and each carbon in the chain is bound to the maximum number of hydrogen atoms. The degree of unsaturation of a fatty acid depends on the number of double or triple bonds present in the hydrocarbon chain. In this respect, a monounsaturated fatty acid contains one double bond, whereas a polyunsaturated fatty acid contains two or more double bonds (see, e.g., Oxford Dictionary of Biochemistry and Molecular Biology, rev. ed., A. D. Smith (ed.), Oxford University Press (2000), and Molecular Biology of the Cell, 3^{rd} ed., B. A. Alberts (ed.), Garland Publishing, New York (1994)).

The fatty acid chains of the inventive cationic cardiolipin, whether short or long, also can be saturated or unsaturated. Preferred fatty acids range from carbon chain lengths of about C₁ to C₃₂, preferably between about C₄ and about C₂₄, and include tetranoic acid (C_{4:0}), pentanoic acid (C_{5:0}), hexanoic acid (C_{6:0}), heptanoic acid (C_{7:0}), octanoic acid (C_{8:0}), nonanoic acid (C_{9:0}), decanoic acid (C_{10:0}), undecanoic acid (C_{11:0}), dodecanoic acid (C_{12:0}), tridecanoic acid (C_{13:0}), tetradecanoic (myristic) acid (C_{14:0}), pentadecanoic acid (C_{15:0}), hexadecanoic (palmatic) acid (C_{16:0}), heptadecanoic acid (C_{17:0}), octadecanoic (stearic) acid (C_{18:0}), nonadecanoic acid (C_{19:0}), eicosanoic (arachidic) acid (C_{20:0}), heneicosanoic acid (C_{21:0}), docosanoic (behenic) acid (C_{22:0}), tricosanoic acid (C_{23:0}), tetracosanoic acid (C_{24:0}), 10-undecenoic acid (C_{11:1),} 11-dodecenoic acid (C_{12:1}), 12-tridecenoic acid (C_{13:1}), myristoleic acid (C_{14:1}), 10-pentadecenoic acid (C_{15:1}), palmitoleic acid (C_{16:1}), oleic acid (C_{18:1}), linoleic acid (C_{18:2}), linolenic acid (C_{18:3}), eicosenoic acid (C_{20:1}), eicosdienoic acid (C_{20:2}), eicosatrienoic acid (C_{20:3}), arachidonic acid (cis-5,8,11,14-eicosatetraenoic acid), and cis-5,8,11,14,17-eicosapentaenoic acid, among others. For ether analogs, the alkyl chain will also range from C₁ to C₃₂ preferably between about C₄ and about C₂₄. Other fatty acid chains also can be employed as R₁ and/or R₂, R₃ and/or R₄ substituents. Examples of such include saturated fatty acids such as ethanoic (or acetic) acid, propanoic (or propionic) acid, butanoic (or butyric) acid, hexacosanoic (or cerotic) acid, octacosanoic (or montanic) acid, triacontanoic (or melissic) acid, dotriacontanoic (or lacceroic) acid, tetratriacontanoic (or gheddic) acid, pentatriacontanoic (or ceroplastic) acid, and the like; monoethenoic unsaturated fatty acids such as trans-2-butenoic (or crotonic) acid, cis-2-butenoic (or isocrotonoic) acid, 2-hexenoic (or isohydrosorbic) acid, 4-decanoic (or obtusilic) acid, 9-decanoic (or caproleic) acid, 4-dodecenoic (or linderic) acid, 5-dodecenoic (or denticetic) acid, 9-dodecenoic (or lauroleic) acid, 4-tetradecenoic (or tsuzuic) acid, 5-tetradecenoic (or physeteric) acid, 6-octadecenoic (or petroselenic) acid, trans-9-octadecenoic (or elaidic) acid, trans-11-octadecenoic (or vaccinic) acid, 9-eicosenoic (or gadoleic) acid, 11-eicosenoic (or gondoic) acid, 11-docosenoic (or cetoleic) acid, 13-decosenoic (or erucic) acid, 15-tetracosenoic (or nervonic) acid, 17-hexacosenoic (or ximenic) acid, 21-triacontenoic (or lumequeic) acid, and the like; dienoic unsaturated fatty acids such as 2,4-pentadienoic (or β-vinylacrylic) acid, 2,4-hexadienoic (or sorbic) acid, 2,4-decadienoic (or stillingic) acid, 2,4-dodecadienoic acid, 9,12-hexadecadienoic acid, cis-9, cis-12-octadecadienoic (or α-linoleic) acid, trans-9, trans-12-octadecadienoic (or linlolelaidic) acid, trans-10,trans-12-octadecadienoic acid, 11,14-eicosadienoic acid, 13,16-docosadienoic acid, 17,20-hexacosadienoic acid and the like; trienoic unsaturated fatty acids such as 6,10,14-hexadecatrienoic (or hiragonic) acid, 7,10,13-hexadecatrienoic acid, cis-6, cis-9-cis-12-octadecatrienoic (or γ-linoleic) acid, trans-8, trans-10-trans-12-octadecatrienoic (or β-calendic) acid, cis-8, trans-10-cis-12-octadecatrienoic acid, cis-9, cis-12-cis-15-octadecatrienoic (or α-linolenic) acid, trans-9, trans-12-trans-15-octadecatrienoic (or α-linolenelaidic) acid, cis-9, trans-11-trans-13-octadecatrienoic (or α-eleostearic) acid, trans-9, trans-11-trans-13-octadecatrienoic (or α-eleostearic) acid, cis-9, trans-11-cis-13-octadecatrienoic (or punicic) acid, 5,8,11-eicosatrienoic acid, 8,11,14-eicosatrienoic acid and the like; tetraenoic unsaturated fatty acids such as 4,8,11,14-hexadecatetraenoic acid, 6,9,12,15-hexadecatetraenoic acid, 4,8,12,15-octadecatetraenoic (or moroctic) acid, 6,9,12,15-octadecatetraenoic acid, 9,11,13,15-octadecatetraenoic (or α- or β-parinaric) acid, 9,12,15,18-octadecatetraenoic acid, 4,8,12,16-eicosatetraenoic acid, 6,10,14,18-eicosatetraenoic acid, 4,7,10,13-docasatetraenoic acid, 7,10,13,16-docosatetraenoic acid, 8,12,16,19-docosatetraenoic acid and the like; penta- and hexa-enoic unsaturated fatty acids such as 4,8,12,15,18-eicosapentaenoic (or timnodonic) acid, 4,7,10,13,16-docosapentaenoic acid, 4,8,12,15,19-docosapentaenoic (or clupanodonic) acid, 7,10,13,16,19-docosapentaenoic, 4,7,10,13,16,19-docosahexaenoic acid, 4,8,12,15,18,21-tetracosahexaenoic (or nisinic) acid and the like; branched-chain fatty acids such as 3-methylbutanoic (or isovaleric) acid, 8-methyldodecanoic acid, 10-methylundecanoic (or isolauric) acid, 11-methyldodecanoic (or isoundecylic) acid, 12-methyltridecanoic (or isomyristic) acid, 13-methyltetradecanoic (or isopentadecylic) acid, 14-methylpentadecanoic (or isopalmitic) acid, 15-methylhexadecanoic, 10-methylheptadecanoic acid, 16-methylheptadecanoic (or isostearic) acid, 18-methylnonadecanoic (or isoarachidic) acid, 20-methylheneicosanoic (or isobehenic) acid, 22-methyltricosanoic (or isolignoceric) acid, 24-methylpentacosanoic (or isocerotic) acid, 26-methylheptacosanoic (or isomonatonic) acid, 2,4,6-trimethyloctacosanoic (or mycoceranic or mycoserosic) acid, 2-methyl-cis-2-butenoic(angelic) acid, 2-methyl-trans-2-butenoic (or tiglic) acid, 4-methyl-3-pentenoic (or pyroterebic) acid and the like.

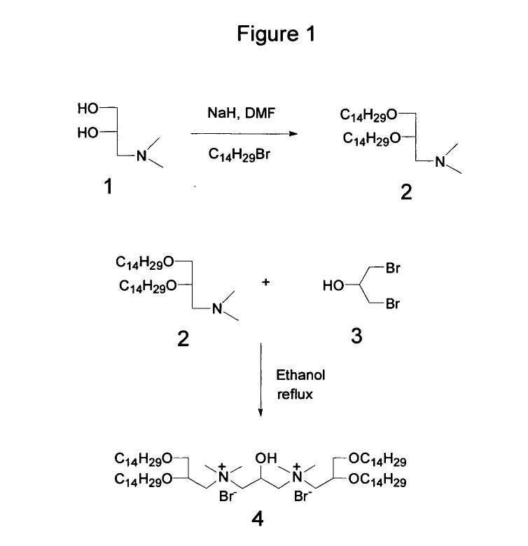

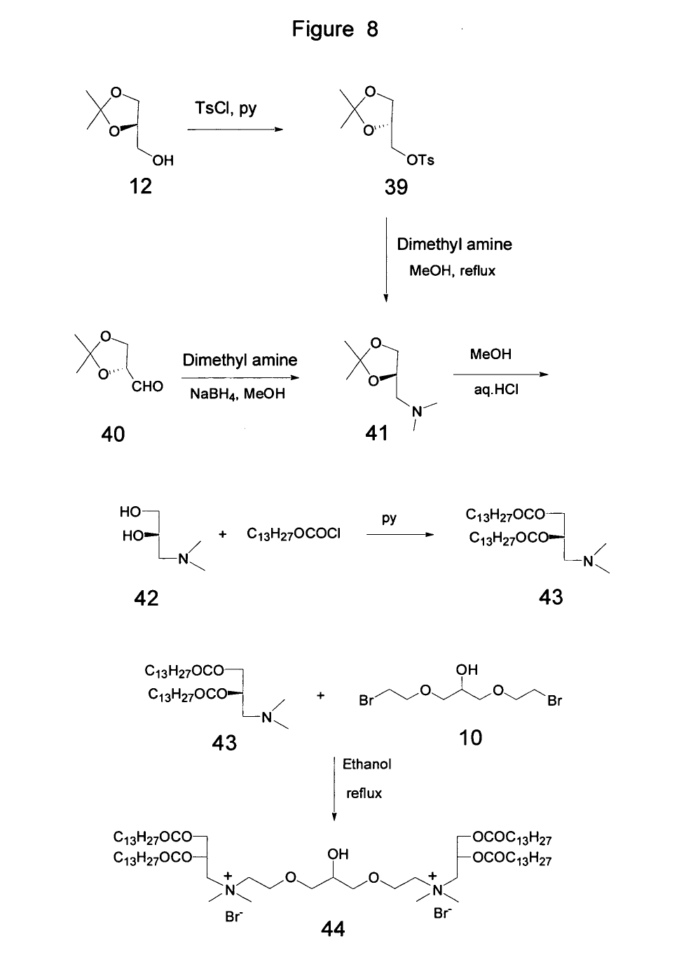

The inventive cationic cardiolipin molecules can be made in accordance with any suitable method, such as are known in the art. However, typically, these fall into one of two general synthetic strategies. In a first route, he hydrocarbon chain becomes connected to the glycerol backbone by ether linkage. This type of synthetic route is exemplified by Examples 1-8 below (FIGS. 1-7). In accordance with a second strategy for synthesizing the inventive cationic cardiolipin, the hydrocarbon chain becomes connected to the glycerol backbone by ester linkage. This type of synthetic route is described below in Example 9 (FIG. 8). It will be understood by the skilled artisan that, where the synthesis of particular compounds is described in the Examples, similar reaction schemes can be employed to construct the inventive cationic cardiolipin molecules that differ, for example, in the nature of the R₁, R₂, R₃, R₄ or other substitutents.

However made, the inventive cationic cardiolipin molecules can be included in compositions, such as liposomal formulations, complexes, emulsions, suspensions, etc. Such formulations can be prepared by any suitable technique, depending on the type of composition, which are known to those of ordinary skill in the art.

Such formulations can include ingredients in addition to the inventive cationic cardiolipin, such as one or more co-lipids or physiologically-acceptable carriers. For purposes of definition, the term “co-lipid” refers to any hydrophobic material that may be combined with a cationic cardiolipin, and includes amphipathic lipids, such as phospholipids, and neutral lipids, such as cholesterol and other sterols, as well as lyso forms of such lipids. The co-lipids can be any of the natural or synthetic phospholipids or mono-, di-, or triglycerols. The natural phospholipids are typically those from animal and plant sources, such as phosphatidycholine, phosphatidylethanolamine, sphingomyelin, phosphatidylserine, or phophatidylinositol. Other suitable co-lipids include sterols (such as cholesterol, derivatives of cholesterol, coprostanol, cholestanol, cholestane, cholesterol hemisuccinate, cholesterol sulfate, and mixtures thereof) and tocopherols (e.g., a tocopherol). Other suitable co-lipids for inclusion in the inventive composition include phosphatidylcholines, such as dimyristoylphosphatidyl choline, distearoylphosphatidylcholine, dioleylphosphatidyl choline, dipalmitoyl-phosphatidylcholine, diarachidonoylphosphatidylcholine, egg phosphatidylcholine, soy phosphatidylcholine, hydrogenated soy phosphatidylcholine, and mixtures thereof. Also, other known cationic lipids such as for example, N-[1-(2,3-dioleyloxy)propyl]-N,N,N-trimethylammonium chloride (DOTMA), or 1,2-dioleoyloxy-3-(trimethylammonio)-propane (DOTAP), can be included with the inventive cationic cardiolipin in the composition. Where neutral lipids are included, typically the molar ratio of the cationic lipid species (including the inventive cationic cardiolipin) to the neutral lipid species is between about 9/1 to about 1/9.

In addition to the inventive cationic cardiolipin, the liposomal composition, complex, emulsion and the like can include stabilizers, absorption enhancers, antioxidants, phospholipids, biodegradable polymers and medicinally active agents among other ingredients. In some embodiments, it is preferable for the inventive composition, especially liposomal composition to include a targeting agent, such as carbohydrate or a protein or other ligand that binds to a specific substrate, such as antibodies (or fragments thereof) or ligand that recognize cellular receptors. The inclusion of such agents (such as a carbohydrate or one or more proteins selected from groups of proteins consisting of antibodies, antibody fragments, peptides, peptide hormones, receptor ligands such as an antibody to a cellular receptor and mixtures thereof) can facilitate targeting a liposome to a predetermined tissue or cell type.

While the invention contemplates the inclusion of the cationic cardiolipin in any suitable type of composition, a preferred composition is a liposomal composition or other composition containing lipid vesicles. Such composition can include unilamellar or multilamellar vesicles, or mixtures thereof. Any suitable technique can be employed to produce such a liposomal formulation. For example, lipophilic liposome-forming ingredients, such as phosphatidylcholine, a cationic cardiolipin of the present invention, cholesterol and α-tocopherol can be dissolved or dispersed in a suitable solvent or combination of solvents and dried. Suitable solvents include any non-polar or slightly polar solvent, such as t-butanol, ethanol, methanol, chloroform, or acetone that can be evaporated without leaving a pharmaceutically unacceptable residue. Drying can be by any suitable means such as lyophilization. Hydrophilic ingredients, such as some pharmaceutical agents, preservatives, and other agents, can be dissolved in polar solvents, including water, which can be mixed with the lipid phase either prior to drying or upon reconstitution. Mixing the dried lipophilic ingredients with the hydrophilic mixture can form liposomes. Mixing the polar solution with the dry lipid film can be by any means that strongly homogenizes the mixture. Vortexing, magnetic stirring and/or sonicating can effect the homogenization.

Where active agents are included in the liposomes they can be dissolved or dispersed in a suitable solvent and added to the liposome mixture prior to mixing. Typically hydrophilic active agents will be added directly to the polar solvent and hydrophobic active agents will be added to the nonpolar solvent used to dissolve the other ingredients but this is not required. The active agent could be dissolved in a third solvent or solvent mixture and added to the mixture of polar solvent with the lipid film prior to homogenizing the mixture.

Generally, liposomes can have a net neutral, negative or positive charge. For example, positive liposomes can be formed from a solution containing phosphatidylcholine, cholesterol, and enough stearylamine to overcome the net positive charge of cationic cardiolipin. Now positive liposomes can be prepared from solutions containing phosphatidylcholine, cholesterol, and cationic cardiolipin analogs of the present invention.

The liposomes of the present invention can be multi or unilamellar vesicles depending on the particular composition and procedure to make them. Liposomes can be prepared to have substantially homogeneous sizes in a selected size range, such as about 1 micron or less, or about 500 nm or less, about 200 nm or less, or about 100 nm or less. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane.

Liposomes can be coated with biodegradable polymers such as sucrose, epichlorohydrin, branched hydrophilic polymers of sucrose, polyethylene glycols, polyvinyl alcohols, methoxypolyethylene glycol, ethoxypolyethylene glycol, polyethylene oxide, polyoxyethylene, polyoxypropylene, cellulose acetate, sodium alginate, N,N-diethylaminoacetate, block copolymers of polyoxyethylene and polyoxypropylene, polyvinyl pyrrolidone, polyoxyethylene X-lauryl ether wherein X is from 9 to 20, and polyoxyethylene sorbitan esters.

The liposomal (or other lipid) composition or formulation can be in any desired form. For example, for pharmaceutical use, the composition can be ready for administration to a patient. Where such compositions contain liposomes or other types of lipid vescicles, such formulations typically are in the form of vesicles in an aqueous medium. Alternatively, the formulation can be in dried or lyophilized form, in which instance, the composition preferably includes a cryoprotectant as well. Suitable cryoprotectants include, for example, sugars such as trehalose, maltose, lactose, sucrose, glucose, and dextran, with the most preferred sugars from a performance point of view being trehalose and sucrose. Other more complicated sugars can also be used, such as, for example, aminoglycosides, including streptomycin and dihydrostreptomycin.

Antioxidants can be included in liposomes or other lipid formulations including the inventive cationic cardiolipin. Suitable antioxidants include compounds such as ascorbic acid, tocopherol, and deteroxime mesylate. Absorption enhancers can be included in the lipid formulation. Suitable absorption enhancers include Na-salicylate-chenodeoxy cholate, Na deoxycholate, polyoxyethylene 9-lauryl ether, chenodeoxy cholate-deoxycholate and polyoxyethylene 9-lauryl ether, monoolein, Na tauro-24,25-dihydroflisidate, Na taurodeoxycholate, Na glycochenodeoxycholate, oleic acid, linoleic acid, and linolenic acid. Polymeric absorption enhancers can also be included such as polyoxyethylene ethers, polyoxyethylene sorbitan esters, polyoxyethylene 10-lauryl ether, polyoxyethylene 16-lauryl ether, and azone (1-dodecylazacycloheptane-2-one).

The composition according to the present invention including the inventive cationic cardiolipin can be employed in a variety of applications, such as the in vivo or in vitro delivery of peptides, polypeptides, proteins, nucleotides, polynucleotides, small molecules, or other agents to human or animal (typically vertebrate) patients, to plants, or to cells in culture. The compositions can be used, for example, in any procedure comprising the use of liposomes or lipid vesicles to deliver substances intracellularly either in vitro or in vivo (Felgner et al. U.S. Pat. No. 5,264,618). The compositions also can be used cosmetically, for example, as a dermatological preparation. Accordingly, the composition can be formulated for use depending on the desired end use.

For application to human or animal patients, the composition preferably includes one or more physiologically (or pharmaceutically) acceptable vehicle or carrier. Any such vehicle, such as those commonly employed in lipid formulations, can be employed. Accordingly, the invention provides a pharmaceutical preparation comprising the cationic cardiolipin of the present invention, such as, for example, formulated as described, and a pharmaceutically acceptable carrier or vehicle.

It is believed that compounds according to the present invention can impart beneficial effects on patients and can, for example, combat the effects of aging, cancer, heart disease, etc. Accordingly, the pharmaceutical preparation of the present invention can be used beneficially to treat certain individuals without including additional agents. However, the pharmaceutical preparation of the present invention can include a pharmacologically effective amount of one or more therapeutic agents. In this context, an effective amount of a therapeutic agent is an amount suitable to impart a therapeutic effect upon application of the composition to a human or animal. The effective amount of a given therapeutic agent will depend on the agent, but the determination of an effective amount for inclusion within the inventive composition is within the ordinary skill of the art.

In one embodiment, the pharmaceutical preparation can include, as an active agent, a therapeutically effective nucleoside analog or nucleotide analog. Examples of suitable nucleoside analogs or nucleotide analogs include nucleoside or nucleotide analogs having an antiviral effect, such as dideoxynucleotides, didehydronucleotides, halogenated or azido derivatives of nucleosides, and acyclic nucleosides. For example, nucleoside or nucleotide analogs having halo-substituted purine or pyrimidine rings such as 5-trifluoromethyl-2′-deoxyuridine or 5-flurouracil; nucleoside or nucleotide analogs having halo- and azido substituted ribose moieties, such as 3′-azido-3′deoxythymidine (AZT), nucleoside analogs having carbon substituted for oxygen in the ribose moiety (carbocyclic nucleosides), or nucleotide analogs having an acyclic pentose such as acyclovir or gancyclovir (DHPG) are suitably included in the composition. The liposomal delivery of such analogs is known in the art (Hosteter et al. U.S. Pat. No. 5,223,263), and the inventive compositions can be used suitably to deliver such agents to patients that would benefit from such treatment. The antiviral potency of these analogs is found to be increased when they are presented to the cells as phospholipid derivatives. These derivatives may be incorporated into the liposomal structure for administration to cells thereby forming a more stable liposomal complex, which can deliver greater amounts of drugs to target cells with less toxicity. Effective antiviral lipid derivatives of nucleoside analogs comprise phosphatidyl 2′,3′-dideoxynucleosides, 2′,3′-didehydronucleosides, 3′-azido-2′-deoxynucleosides, 3′-fluorodeoxynucleosides and 3′-fluorodideoxynucleosides, 9-β-D-arabinofuranosyladenine (araA), 1-β-D-arabinofuranosylcytidine (araC), nucleosides such as acyclovir and gancyclovir having an acyclic ribose group, or the same nucleoside analogs as diphosphate diglyceride derivatives. Preferred species of lipid derivatives of antiviral or antiretroviral nucleoside analogs for the treatment of HIV infection using cationic lipid medicated liposomal delivery are 3′-phospholipid derivatives of 3′-azido-2′,3′-dideoxypyrimidine, 3′-halopyrimidine dideoxynucleoside, or a 2′,3′-didehydro-2′,3′-dideoxynucleoside, for example phosphatidyl 3′-azido-3′deoxythymidine (pAZT) or phosphatidyl 2-chlrodeoxyadenosine. Certain viral infections, comprising herpes, cytomegalovirus, and hepatitis B infections are effectively treated with nucleoside analogs comprising acyclovir, gancyclovir, 1-(2-deoxy-2′-fluoro-1-β-D-arabunofuranosyl)-5-iodocytosine (FIAC) or 1(2′-deoxy-2′-fluoro-1-β-D-arabinofuranosyl) -5-iodouracil (FIAU). Phospholipid derivatives of these agents, preferably the phosphatidyl and diphosphate diglyceride derivatives can be administered in these diseases using cationic lipid liposomal delivery systems, according to the invention. Details of the structures, synthesis and liposomal delivery of lipid derivatives of antiviral nucleosides has been reported (Hostetler et al. U.S. Pat. No. 6,448,392), and the inventive compositions can be used suitably to deliver such agents to patients that would benefit from such treatment.

In another embodiment, the pharmaceutical preparation can include, as an active agent, a therapeutically effective amount of a corticosteroid or a norm-steroidal anti-inflammatory agent. Another suitable active agent includes a bioactive lipid, such as, for example, 1-O-octadecyl-2-O-methyl-sn-glycero-3-phosphocholine. Other suitable active agents include drugs, such as anticancer dugs and topical antibiotics such as clindamycin, tobramycin, neomycin, gentamycin, tetracycline, erythromycin; antifingal agents, such as clotrimazole, miconazole, nystain, lactoconazole, econazole, and tolnaftate; retinoic acid for the treatment of acne; and agents for the treatment of herpes simplex and comprising antiviral nucleoside analogs such as acyclovir and gancyolovir. These nucleoside analog formulations preferably comprise lipid derivatives of the antiviral agents, particularly the phosphatidylglycerol derivatives as disclosed in (Felgner et al. U.S. Pat. No. 5,459,127) and as such may be incorporated into liposomes or other formulations comprising one or more cationic cardiolipin analogs of the invention. Examples of other suitable active agents include antifungal agents, oxidants, proteins, polypeptides, and therapeutic polynucleotides. Preferred proteins include, for example, antibodies, such as monoclonal antibodies or fragments thereof.

Therapeutically important polynucleotides suitable for cationic lipid medicated delivery are negatively charged novel oligonucleotides of various technologies, including antisense polynucleotide sequences, useful in eliminating or reducing the production of a gene product (Ts'o et al. 1987). Many of these oligonucleotide species, which are scarce and expensive to synthesize, are inefficiently captured by encapsulation into liposomes of negatively charged lipids, according to ordinary current methods.

Examples of therapeutic polynucleotides include ribozymes, interfering RNA (RNAi) antisense RNA or DNA sequences, which can target desired sequences within cells, such as genes associated with a disease state (e.g., oncogenes or viral genes). A preferred therapeutic polynucleotide for targeting desired genes is a 10 to 30-mer antisense polynucleotide, preferably a 15-mer sequence, such as, for example, targeted against the c-raf gene (see, e.g., U.S. Pat. No. 6,126,965, disclosing a 15-mer anti c-raf-1 oligonucleotide having the sequence 5′-GTGCTCCATTGATGC-3′ (SEQ ID NO: 1)). Where oligonucleotides are included in the composition, they preferably contain one or more phosphothioate linkages preferably two phosphothioate linkages. Most preferably, oligonucleotides for inclusion in the inventive composition contain one phosphothioate linkage at each terminal end, but they can be present anywhere from one end to the other end (e.g., between the ends) of an oligonucleotide. Other preferred genes for targeting with therapeutic riboozymes, interfering RNA (RNAi) antisense RNA or DNA sequences include viral genes, particularly HIV genes, such as the rev transactivator. In other embodiments, a therapeutic polynucleotide can be one which is absent or mutated in a diseased state, or can encode a gene product that is deficient or absent in a diseased state. Other polynucleotides can encode therapeutic polypeptides, such as, for example, immunogenic peptides (which can be used as vaccines), natural hormones, or a synthetic analogue of a natural hormone.

Other suitable active agents for inclusion in the inventive formulation include agents which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, the alimentary and excretory systems, the histamine system and the central nervous system. Suitable agents may be selected from, for example, proteins, enzymes, hormones, nucleotides (including sense and antisense oligonucleotides), polynucleotides, nucleoproteins, polysaccharides, glycoproteins, lipoproteins, polypeptides, and steroids. Active agents can be analgesics, anesthetics, anti-arrythmic agents, antibiotics, antiallergic agents, antifingal agents, anticancer agents, anticoagulants, antidepressants, antidiabetic agents, anti-epilepsy agents, anti-inflammatory corticosteroids, agents for treating Alzheimers or Parkinson's disease, antiulcer agents, anti-protozoal agents, anxiolytics, thyroids, anti-thyroids, antivirals, anoretics, bisphosphonates, cardiac inotropic agents, cardiovascular agents, corticosteroids, diuretics, dopaminergic agents, gastrointestinal agents, hemostatics, hypercholesterol agents, antihypertensive agents (e.g., dihydropyridines), antidepressants and cox-2 inhibitors, immunosuppressive agents, anti-gout agents, anti-malarials, steroids, terpinoids, triterpines, retinoids; anti-ulcer H₂-receptor antagonists, hypoglycemic agents, moisturizers, cosmetics, anti-migraine agents, antimuscarinic agents, antiinflammatory agents, such as agents for treating rheumatology, arthritis, psoriasis, inflammatory bowel disease, Crohn's disease; or agents for treating demyelinating diseases including multiple sclerosis, ophthalmic agents, vaccines (e.g., against pneumonia, hepatitis A, hepatitis B, hepatitis C, cholera toxin B subunit, influenza virus, typhoid, plasmodium falciparun, diptheria, tetanus, HSV, tuberculosis, HIV, SARS virus, pordetela pertussis, measles, mumps and rubella vaccine (MMV), bacterial toxoids, vaccinea virus, adenovirus, canary, polio virus, bacillus calmette guerin (BCG), klebsiella pneumonia, etc.), histamine receptor antagonists, hypnotics, kidney protective agents, lipid regulating agents, muscle relaxants, neuroleptics, neurotropic agents, opioid agonists and antagonists, parasympathomimetics, protease inhibitors, prostglandins, sedatives, sex hormones (e.g., estrogen, androgen), stimulants, sympathomimetics, vasodilators and xanthins and synthetic analogs of these species. The therapeutic agents can be nephrotoxic, such as cyclosporins and amphotericin B, or cardiotoxic, such as amphotericin B and paclitaxel. Exemplary anticancer agents include melphalan, chlormethine, extramustinephosphate, uramustine, ifosfamide, mannomustine, trifosfamide, streptozotocin, mitobronitol, mitoxantrone (see, e.g., published international patent application WO 02/32400), methotrexate, fluorouracil, cytarabine, tegafur, idoxide, taxanes (e.g., taxol, paclitaxel, etc.) (see, e.g., published international patent application WO 00/01366), daunomycin, daunorubicin, bleomycin, amphotericin, carboplatin, cisplatin, paclitaxel, BCNU, vincristine, camptothecin and derivatives thereof (e.g., SN38 (see, e.g., published international patent application WO 02/058622, irinotecan (see, e.g., published international patent application WO 03/030864)), antracyclines, antibodies, cytoxines, doxorubicin, etopside, cytokines, ribozymes, interferons, oligonucleotides and functional derivatives of the foregoing. Additional examples of drugs which may be delivered according to the method include, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-(S)-estradiol, ethinyl estradiol, ethinyl estradiol 3-methyl ether, prednisolone, 17-α-hydroxyprogesterone acetate, 19-norprogesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, vinorelbine (see, e.g., published international patent application WO 03/018018) diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinopril, enalapril, enalaprilat captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, colchicine, glucagon, thyroid stimulating hormone, parathyroid and pituitary hormones, digestive hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons (e.g., consensus interferon, interferon α-2a, interferon α-2b, α-, β, or γ-interferons), interleukins, growth hormones such as human growth hormone and its derivatives such as methione-human growth hormone and des-phenylalanine human growth hormone, bovine growth hormone and porcine growth hormone, fertility inhibitors such as the prostaglandins, fertility promoters, growth factors such as insulin-like growth factor, coagulation factors, pancreas hormone releasing factor, analogs and derivatives of these compounds, and pharmaceutically acceptable salts of these compounds, or their analogs or derivatives. The therapeutic agent can be a mixture of agents (e.g., two or more agents) that can be beneficially co-administered in the liposome formulation.

The pharmaceutical formulations can be used to deliver therapeutic agents by various routes and to various sites in the animal body to achieve a desired therapeutic effect. Local or systemic delivery of the therapeutic agent can be achieved by administration comprising application or insertion of the formulation into body cavities or tissues, oral delivery, inhalation or insufflation of an aerosol, topical administration to skin or mucosal surfaces, or by parenteral introduction, comprising intramuscular, intravenous, intratumoral, intradermal, peritoneal, subcutaneous and topical administration. The effect of the cationic lipids in these formulations is to enhance the potency and efficiency of the therapeutic agent contained therein by facilitating its intracellular delivery (Felgner et al. U.S. Pat. No. 5,459,127). Accordingly, the invention provides a method of delivering one or more active agents by administering a composition, as herein described, comprising a cationic cardiolipin and one or more active agents. The method can be used therapeutically—i.e., to deliver one or more active agents to a human or animal patient in need of therapy (or to a plant), or the method can be used to deliver one or more active agents to cells in culture. The method can be used to administer virtually any active agent. For therapeutic use, it is thought to be general for active agents that are stable in the presence of surfactants. Hydrophilic active agents are suitable and can be included in the interior of the liposomes such that the liposome bilayer creates a diffusion barrier preventing it from randomly diffusing throughout the body. Hydrophobic active agents are thought to be particularly well suited for use in the present method because they not only benefit by exhibiting reduced toxicity but they tend to be well solubilized in the lipid bilayer of liposomes.

Chemotherapeutic agents are well suited for use in the method. Liposomal or other lipid formulations containing chemotherapeutic agents may be injected directly into the tumor tissue for delivery of the chemotherapeutic agent directly to cancer cells. In some cases, particularly after resection of a tumor, the liposome formulation can be implanted directly into the resulting cavity or may be applied to the remaining tissue as a coating. In cases in which the liposome formulation is administered after surgery, it is possible to utilize liposomes having larger diameters of about 1 micron since they do not have to pass through the vasculature.

A particularly preferred embodiment involves a formulation for treatment or prevention of herpes simplex and the use of such a formulation for the treatment of prevention of herpes infection. As noted herein, such a formulation desirably includes, in addition to the inventive cationic cardiolipin, a pharmacologically effective concentration of acyclovir, gancyclovir, 1-(2-deoxy-2′-fluoro-1-β-D-arabinofuranosyl)-5-iodocytosine (FIAC) or 1(2′-deoxy-2′-fluoro-1-β-D-arabinofuranosyl)5-iodouracil (FIAU). The composition can be administered to an individual infected with herpes simplex or at risk for infection with herpes simplex, for example, by topical administration (desirably to mucosal tissue).

The invention provides a method for the administration of pharmaceutical preparations, which in addition to liposome formulations of active agents include non-toxic, inert pharmaceutically suitable excipients. Pharmaceutically suitable excipients include solid, semi-solid or liquid diluents, fillers and formulation auxiliaries of all kinds. The invention also includes pharmaceutical preparations in dosage units. This means that the preparations are in the form of individual parts, for example vials, syringes, capsules, pills, suppositories, or ampoules, of which the content of the liposome formulation of active agent corresponds to a fraction or a multiple of an individual dose. The dosage units can contain, for example, 1, 2, 3, or 4 individual doses, or ½, ⅓, or ¼ of an individual dose. An individual dose preferably contains the amount of active agent which is given in one administration and which usually corresponds to a whole, a half, a third, or a quarter of a daily dose.

Tablets, dragees, capsules, pills, granules, suppositories, solutions, suspensions and emulsions, pastes, ointments, gels, creams, lotions, powders and sprays can be suitable pharmaceutical preparations. Suppositories can contain, in addition to the liposomal active agent, suitable water-soluble or water-insoluble excipients. Suitable excipients are those in which the inventive liposomal active agent is sufficiently stable to allow for therapeutic use, for example polyethylene glycols, certain fats, and esters or mixtures of these substances. Ointments, pastes, cream, and gels can also contain suitable excipients in which the liposomal active agent is stable.

The active agent or its pharmaceutical preparations can be administered to the patient or to cells in vivo, such as intravenously, subcutaneously, locally, topically (e.g., to skin or dermal tissue, or to mucosal tissue), orally, parenterally, intraperitoneally, and/or rectally or by direct injection into tumors or sites in need of treatment by such methods as are known or developed. Moreover, in some embodiments, it is desirably to administer the pharmaceutical preparation including the inventive cationic cardiolipin and, optionally, an active agent to the cells of a patient in vitro, and thereafter to return the cells to the patient. Such treatment can be effective, for example, in instances wherein gene expression in the re-introduced cells can be effective in combating a disease in the patient, such as, for example, in the treatment of cancers.

The inventive cationic cardiolipin, and the formulations noted above, facilitate a method of treating a disease in a vertebrate (such as a human or non-human animal), comprising the step of administering a pharmaceutical preparation as described herein, which typically includes a therapeutic agent specific for the treatment of the disease, to the patient. In accordance with the inventive method, a preparation as herein described (desirably containing an active agent) is administered to a vertebrate in need of treatment in an amount and at a location sufficient to treat the disease within the vertebrate. In some embodiments, the therapeutic agent will become incorporated into at least one cell of the patient, wherein it will exert its effects to treat the disease (such as, for example, where there therapeutic agent is DNA or RNA which is expressed after it is taken up into a cell). In other embodiments, the agent will act extracellularly within the patient to combat the disease. The pharmaceutical preparation is administered to the patient in the manner appropriate to the type of formulation (e.g., dermally to skin or mucosal surfaces, parenteral injection or injection into the body cavity or into tissues, oral administration, etc.). It should be realized that the effective treatment of a disease, in accordance with the inventive methods, while desirably eliminates the disease or its symptoms, need not completely eradicate the effects of the disease. Indeed, successful therapy in accordance with the inventive method can be measured by a reduction in the severity of a disease, infection, or a reduction in the rate by which a disease progresses within a patient.

In one embodiment, the method disease is cancer, in which instance, the pharmaceutical preparation can comprise a suitable anticancer agent, such as a chemotherapeutic agent (e.g., an anticancer drug) as herein described or a polynucleotide selected from the group consisting of ribozymes, interfering RNA (RNAi) and antisense RNA or DNA sequences. A preferred anticancer polynucleotide is a c-raf antisense oligonucleotide. In another embodiment, the disease is a viral infection, such as herpes simplex or HIV. For treatment of viral infections, the pharmaceutical preparation typically includes an antiviral agent, such as described herein. For example, for treating HSV, the preparation can include acyclovir, gancyclovir, 1-(2-deoxy-2′-fluoro-1-β-D-arabinofuranosyl)-5-iodocytosine (FIAC) or 1(2′-deoxy-2′-fluoro-1-β-D-arabinofuranosyl)₅-iodouracil (FIAU). For treating HIV, the composition can include an antiviral nucleoside, such as 3′-azido-3′-deoxythymidine (AZT).

The invention also provides a method of introducing an active agent into a cell or cells. In accordance with the inventive method, a composition comprising a cationic cardiolipin as described herein is prepared that also can include the active agent. The cell then is contacted with the agent such that the cell takes up the active agent. Alternatively, the composition can be prepared that does not include the active agent, and the cell can be contacted with the composition in the presence of the active agent such that the active agent is taken up into the cell.

The cell or cells can be an in vitro cell culture. Where the cell or cells are in vitro, the composition comprising the cationic cardiolipin and/or the active agent can be delivered to the cells by admixing it with the culture medium. Alternatively, the cell or cells can be in vivo, within a plant or animal (eg., a human) host. In such instances, the composition can be formulated and delivered as herein described.

Where the active agent is a polynucleotide, the inventive method can be used to transfect the cell or cells with the polynucleotide. The method can be employed to transfect cells in vitro (e.g., in culture), or to deliver therapeutic or diagnostic polynucleotides to cells in vivo. For example, the method can be used to deliver genes to cells in culture or to patients in connection with a gene therapy regimen. For therapeutic application, the invention, thus, provides a method of gene therapy comprising administering a pharmaceutical composition comprising one or more nucleic acids to a patient in need of treatment, wherein the composition comprises cationic cardiolipinIn this respect, the polynucleotide can be an expression construct encoding a gene, which is expressed within the cell after transfection in accordance with the inventive method. For expression, such a gene construct desirably includes suitable regulatory elements to facilitate expression. Thus, the expression construct typically includes, in addition to the coding sequence, a promoter in operable linkage with the coding sequence. Also, the coding sequence can include other regulatory elements, such as ribosome entry sites, enhances, etc. The construction of expression constructs is within the ordinary skill of the art.

Contemplated uses comprise transfection procedures corresponding to those presently known and using amphipathic lipids, including commercial cationic lipid preparations, such as Lipofectin™, and using conventional cationic lipid technology and methods. Accordingly, the lipid compositions disclosed herein can be used to facilitate the intercellular delivery of DNA or mRNA sequences coding for therapeutically active polypeptides (Felgner et al. U.S. Pat. No. 5,459,127). They can be similarly used for the liposomal delivery of the expressed gene product, the polypeptide or protein itself. Thus cationic lipid mediated delivery of DNA and mRNA polynucleotides or proteins can provide therapy for genetic disease by supplying deficient or absent gene products to treat any genetic disease in which the defective gene and or its product has been identified, such as Duchenne's dystrophy (Kunkel et al. 1989).

The transfection procedures and kits described above may be applied by direct injection of cationic lipids together with DNA, RNA or proteins into cells of an animal in vivo. However, it has been recently shown that cationic lipids are particularly effective at facilitating in vitro transfection of cells. Therefore, the above therapies can be alternatively carried out by in vitro transfection of some of the cells of an animal using cationic lipid delivery methods, and reintroduction of the cells into the animal. The ability to transfect cells at high efficiency with cationic lipids thus provides an alternate method for immunization. The gene for an antigen is introduced by means of cationic lipid-mediated delivery, into cells that have been removed from an animal. The transfected cells, now expressing the antigen, are reinjected into the animal where the immune system can now respond to the endogenous antigen. The process can be enhanced by co-injection of either an adjuvant or lymphokines, or a gene coding for such lymphokines, to further stimulate the lymphoid cells (Felgner et al. U.S. Pat. No. 5,459,127).

The invention also provides a kit for transfection of polynucleotides into cells. The kit includes cationic cardiolipin as described herein, and it also can include a desired polynucleotide for transfection. Where present, the polynucleotide can be packaged separately or included with the cationic cardiolipin, for example in a preparation as herein described. The kit also can include instructions for using the kit to facilitate transfection. The instructions can include, for example, instructions for formulating the polynucleotide and the cationic cardiolipin into a preparation that can be used to transfect cells. The kit also can include reagents for facilitating transfection, such as buffers, culture medium, etc. Also, the kit can include containers for storing the cationic cardiolipin, for storing the reagents, for storing a preparation including the cationic cardiolipin and polynucleotide, or containers for preparing the preparation. Also, the kit can include materials to facilitate transfection, such as pipettes or pipette tips, culture dishes or bottles, or other suitable materials.

The invention also is directed to methods of delivering active agents to cells. The methods can be carried out by preparing liposomes that include active agents and cationic cardiolipin variants/analogs as synthesized by the above-disclosed methods. The liposomes are then delivered to a cell. This can be carried out by adding the liposomes to the cell culture medium.

The following examples further illustrate the invention but, of course, should not be construed as in any way limiting its scope.

### EXAMPLES

### Example 1

### Synthesis of Cationic Cardiolipin Analog (4) [FIG. 1]

### (R,S)-1,2-Bis-(tetradecyloxy)-3-dimethylamino propane (2)

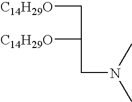

To a suspension of sodium hydride (60% in oil) (6.7 g, 167.92 mmol) and DMF (100 mL) in a three neck round bottom flask was added a solution of 1-dimethylamino-2,3-propanediol (1) (5 g, 41.98 mmol) in DMF (25 mL) at 0° C. dropwise over a period of 1.5 h, then allowed to attain room temperature and stirred for 1 h. Tetradecyl bromide (46.5 g, 167.27 mmol) was added dropwise to the reaction mixture over 90 min. and then allowed to attain room temperature and stirred for 1 h. The reaction mixture temperature was gradually increased to 82° C. and stirred for 24 h at the same temperature. The reaction mixture was cooled to 0° C., added few drops of cold ice water very slowly, then this mixture diluted with water (750 mL). The aqueous layer was extracted with hexane several times (6×150 mL). The organic layer was dried over sodium sulfate. The crude compound was purified by column chromatography over a silica gel (70-230 mesh) with 20% ethyl acetate in hexane to obtain 1,2-bis-(tetradecyloxy)-3-dimethylamino propane (2) (13 g, 62%) as colorless oil (Wheeler et al. 1987). TLC (SiO₂) methanol/chloroform (1:9) R_{f}˜0.51 ¹H NMR (CDCl₃, 500 MHz): δ 0.88 (t, J=6.8 Hz, 6H), 1.25 (s, 44H), 1.54-1.56 (m, 4H), 2.25 (s, 6H, N—CH₃), 2.26-2.41 (m, 2H, CH₂N), 3.43-3.52 (m, 7H).

### (R,S)-1,3-Bis-(1,2-ditetradecyloxypropyl-3-N,N-dimethyl ammonium bromide)propane-2-ol (4)

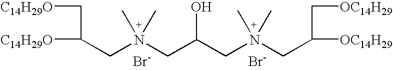

A solution of 1,2-bis-(tetradecyloxy)-3-dimethylamino propane (2) (4.3 g, 84.0 mmol) and 1,3-dibromo glycerol (3) (0.73 g, 3.36 mmol) in anhydrous ethanol (100 mL) (Bhattacharya et al. 1999) was refluxed for a period of 5 days. The reaction mixture was cooled and the solvent was evaporated to give a crude waxy solid. The crude compound was dissolved in hexane (200 mL) and stirred at room temperature for 6 h, kept at 0° C. overnight. The separated solid was filtered and washed with hexane (8×10 mL) to remove the starting material 1,2-bis-(tetradecyloxy)-3-dimethylamine propane. The crude compound was subjected to column chromatography (silica-gel, 70-230 mesh,) eluting with 1-6% methanol in dichloromethane to obtain cationic cardiolipin analog (4) (3.2 g, 77%) as a white solid. TLC (SiO₂) methanol/chloroform (1:9) R_{f}˜0.11. ¹H NMR (CDCl₃, 500 MHz): δ 0.88 (t, J=6.8 Hz, 12H), 1.24-1.31 (m, 88H), 1.56-1.60 (m, 8H), 3.42-3.68 (m, 25H), 3.79-3.85 (m, 4H), 4.39 (t, J=6.8 Hz, 2H), 4.53-4.60 (m, 2H), 5.18-5.24 (m, 1H), 6.50-6.61 (m, 1H). ¹³C NMR (CDCl₃, 125 MHz): δ 14.03, 22.60, 29.28, 29.35, 29.37, 29.39, 29.52, 29.55, 29.57, 29.58, 29.61, 29.62, 29.98, 30.0, 31.84, 52.74, 52.89, 53.16, 54.85, 54.89, 55.01, 62.28, 62.57, 65.02, 68.53, 68.63, 68.88, 69.29, 69.39, 69.80, 70.02, 72.02, 72.82, 72.87. IR (cm⁻¹): 3407 (br), 3223 (br), 2956 (s) H, 2920 (s), 1633, 1467, 1377, 1116, 971, 893, 720. ESI-MS 1162.5 [M+1-Br], 1080.6 [M+1-2Br], 540.7 [M+1-2Br/2]. Mol. Formula C₆₉H₁₄₄Br₂N₂O₅; Elemental analysis; calcd. C, 66.74; H, 11.69; N, 2.26; Br, 12.87; found C, 65.85; H, 11.52; N, 2.31; Br, 13.21.

### Example 2

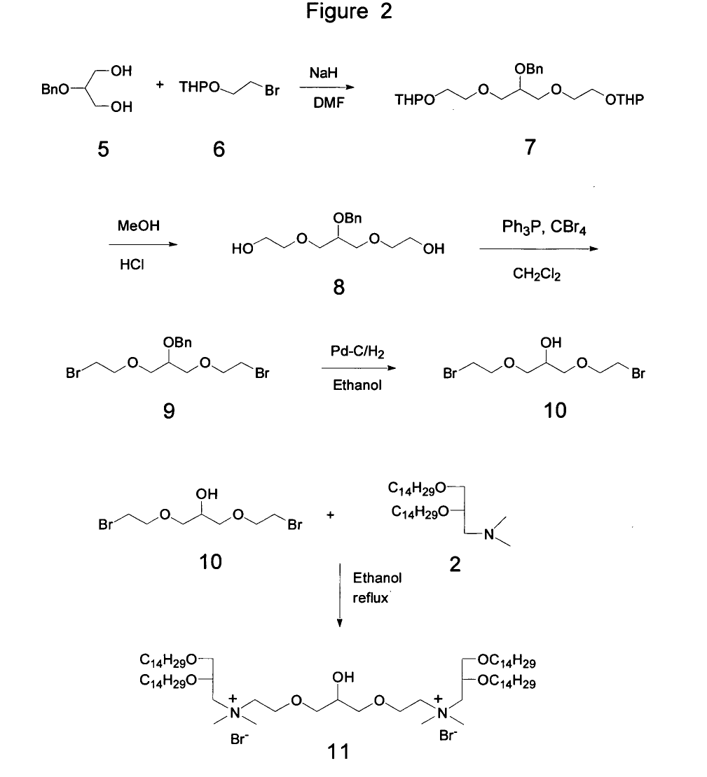

### Synthesis of Cationic Cardiolipin Analog (11) [FIG. 2]

### 1,3 Bis [(2-ethoxy tetrahydro-2H-pyran)]-2-benzyloxy-glycerol (7)

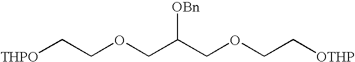

To a stirred suspension of sodium hydride (59.3 g, 1.48 mol, 60% in oil) in anhydrous DMF (300 mL) under argon atmosphere at 0° C., a solution of 2-benzyloxy 1,3-propanediol (5) (90 g, 0.49 mol) in DMF (700 mL) was added over a period of 2 h maintaining the temperature below 15° C. After stirring at room temperature for 2 h, 2-(2-bromoethoxy) tetrahydro-2H-pyran (6) (310 g, 1.48 mol) was added at 0° C. over a period of 3 h maintaining the temperature below 10° C. The reaction mixture was stirred at room temperature for 12 h. The reaction mixture was cooled to 0° C. and ice water was added very slowly to quench excess sodium hydride. The reaction mixture was concentrated under reduced pressure to remove maximum DMF and the crude solution was diluted with water (1 L) and extracted with ethyl acetate (2×500 mL). The organic layer was washed with aqueous saturated sodium chloride (500 mL) and dried over sodium sulfate. The solvent was concentrated under reduced pressure. The crude compound was purified by column chromatography over a silica gel (230-400 mesh) with 10-30% ethyl acetate in hexane to obtain 1,3-bis-[(2-ethoxy tetrahydro-2H-pyran)]-2-benzloxy-glycerol (7) (154 g, 71%) as colorless oil. TLC (SiO₂) hexane/ethyl acetate (3:2) R_{f}˜0.40. ¹H NMR (CDCl₃, 300 MHz): δ 1.41-1.82 (m, 12H), 3.41-3.98 (m, 17H), 4.61 (brs, 2H), 4.78 (s, 2H, OCH₂Ph), 7.24-7.45 (m, 5H, Ph-H).

### 3,7-Dioxa-5-benzyloxy-19-nonanediol (8)

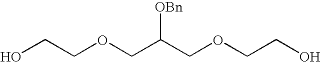

To a solution of 1,3-bis [(2-ethoxy tetrahydro-2H-pyran)]-2-benzyloxy-glycerol (7) (50 g, 0.11 mol) in methanol (500 mL) was added 1N HCl in ether (5 mL) and stirred at room temperature for 2 h. The reaction mixture was neutralized with sodium bicarbonate until the solution became neutral. The reaction mixture was filtered and concentrated under reduced pressure. The crude compound was dissolved in ethyl acetate (1 L), washed with water (100 mL) and dried over sodium sulfate. The organic layer was concentrated under reduced pressure and the crude compound was purified by column chromatography over a silica gel (70-230 mesh) eluting with ethyl acetate and followed by 5% methanol in ethyl acetate to obtain 3,7-dioxa-5-benzyloxy-1,9-nonanediol (8) (27 g, 88%) as colorless oil. TLC (SiO₂) ethyl acetate Rf˜0.10. ¹H NMR (CDCl₃, 300 MHz): δ 3.01 (brs, 2H, OH), 3.50-3.81 (m, 13H), 4.68 (s, 2H, OCH₂Ph), 7.21-7.42 (m, 5H, Ph-H).

### 1,9-Dibromo-3,7-dioxa-5-benzyloxy-nonane (9)

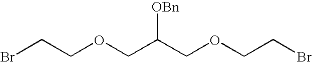

To a solution of 3,7-dioxa-5-benzyloxy-1,9-nonanediol (8) (27 g, 0.1 mol) in anhydrous dichloromethane (400 mL) under argon atmosphere at 0° C., was added triphenylphosphine (65.5 g, 0.25 mol) followed by carbon tetrabromide (79.4 g, 0.24 mol). The reaction mixture was stirred at 0° C. for 2 h. The reaction mixture was diluted with water (300 mL) and the organic layer was separated, dried over sodium sulfate. The organic layer was concentrated under reduced pressure and crude compound was purified by column chromatography over a silica gel (70-230 mesh) with 20% ethyl acetate in hexane to obtain 1,9-dibromo-3,7-dioxa-5-benzyloxy-nonane (9) (36 g, 91%) as colorless oil. TLC (SiO₂) hexane/ethyl acetate (3:2) R_{f}˜0.60. ¹H NMR (CDCl₃, 300 MHz): δ 3.45 (t, J=5.5 Hz, 4H, CH₂Br), 3.60-3.67 (m, 4H, OCH₂), 3.72-3.82 (m, 5H), 4.70 (s, 2H, OCH₂Ph), 7.28-7.38 (m, 5H, Ph-H).

### 1,3-Bis-(2-bromoethoxy) propane-2-ol (10)

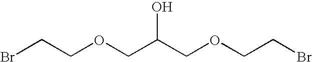

1,9 Dibromo-3,7-dioxa-5-benzyloxy-nonane (9) (36 g, 90.90 mmol) was dissolved in ethanol (110 mL) and hydrogenated with 10% palladium on carbon (3.6 g) for 2 h at a pressure of 50 psi. After filtration of the catalyst, the solution was evaporated under reduced pressure. The crude material was subjected to silica-gel column chromatography (70-230 mesh) and eluted with 60% ethyl acetate in hexane to obtain 1,3-bis-(2-bromoethoxy) propane-2-ol (10) (26 g, 94%) as colorless oil. TLC (SiO₂) hexane/ethyl acetate (1:1) R_{f}˜0.30. ¹H NMR (CDCl₃, 300 MHz): 62.56 (d, J=4.5 Hz, 1H, OH), 3.45 (t, J=6.0 Hz, 4H, CH₂Br), 3.54-3.64 (m, 4H, OCH₂), 3.82 (t, J=5.7 Hz, 4H, OCH₂), 3.96-4.02 (m, 1H).

### (R,S)-1,3-Bis-(1,2-ditetradecyloxypropyl-3-N,N-dimethyl-3-ethoxy ammonium bromide)propane-2-ol (11)

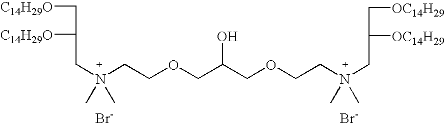

A solution of 1,2-bis-tetradecyloxy-3-dimethylamino propane (2) (50.2 g, 98.36 mmol) and 1,3-bis-(2-bromoethoxy) propane-2-ol (10) (10 g, 32.78 mmol) in anhydrous ethanol (600 mL) was refluxed at 78-80° C. over a period of 5 days. The reaction mixture was cooled and the solvent was evaporated to give a crude waxy solid. Hexane (400 mL) was added and stirred for 1 h. The solid was filtered and washed with hexane (6×100 mL). The compound was purified by recrystallization [ratio of compound/methanol/acetone (1:3:50)] and kept at −20° C. overnight. The separated solid was filtered and washed with cold acetone. The recrystallization was repeated two times to get analytically pure sample. The compound was dried for 24 h under vacuum and then over P₂O₅ for 36 h to obtain cationic cardiolipin analog (11) (30 g, 69%) as a white solid. TLC (SiO₂) methanol/chloroform (1:9) R_{f}˜0.11. ¹H NMR (CDCl₃, 300 MHz): δ 0.88 (t, J=6.7 Hz, 12H), 1.25 (s, 88H), 1.52-1.71 (m, 8H), 3.41-3.68 (m, 29H), 3.95-4.19 (m, 14H), 4.66 (brs, 1H). ¹³C NMR (CDCl₃, 125 MHz): δ 13.92, 22.50, 25.89, 26.03, 29.12, 29.27, 29.32, 29.42, 29.48, 29.50, 29.54, 29.87, 31.74, 52.95, 53.02, 53.50, 65.02, 65.07, 66.57, 68.63, 68.84, 69.15, 71.80, 72.65, 73.29. IR (cm⁻¹): 3323 (br), 2918 (s), 2873 (s), 1468 (s), 1123 (brs). ESI-MS 1248.6 [M+1-Br], 584.3 [M+1-2Br/2]. Mol. Formula C₇₃H₁₅₂Br₂N₂O₇; elemental analysis; calcd. C, 65.93; H, 11.52, N, 2.11, Br, 12.02; found C, 65.93; H, 11.40; N, 2.11; Br, 11.99.

### Example 3

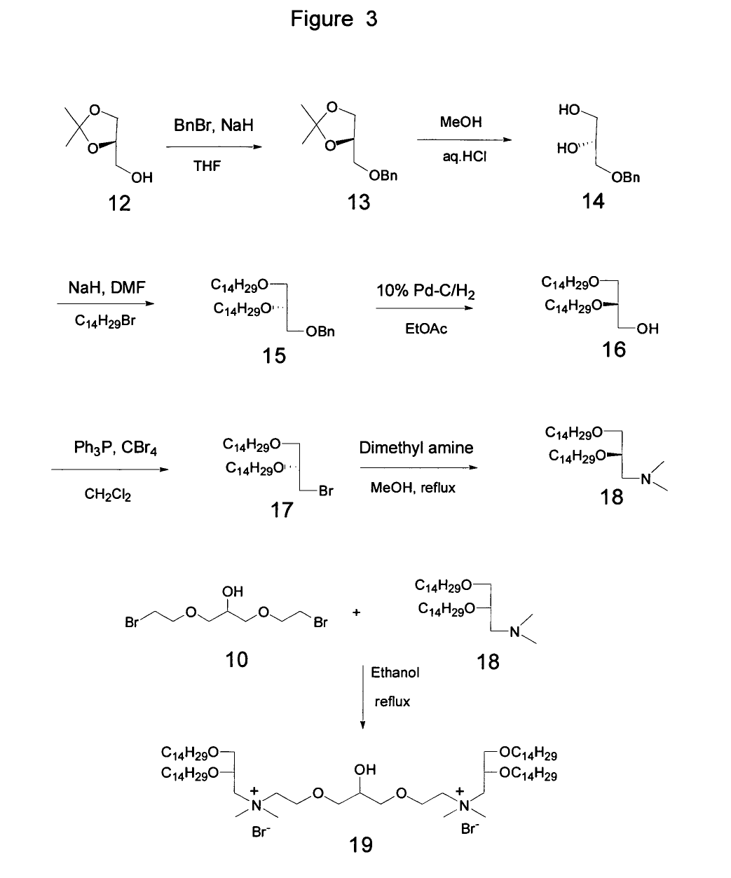

### Synthesis of Cationic Cardiolipin Analog (19) [FIG. 3]

### (R)-4-(Benzyloxymethyl)-2,2-dimethyl-1,3-dioxolane (13)

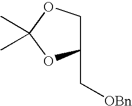

To a stirred suspension of sodium hydride (30.3 g, 0.75 mol, 60% in oil) in anhydrous terahydrofuran (500 mL) under argon atmosphere at 0° C., was added R (−)-2,2-dimethyl-1,3-dioxolane-4-methanol (12) (50 g, 0.37 mol) over period of 1 h maintaining the internal temperature below 20° C. After stirring at room temperature for 1 h, benzyl bromide (97.1 g, 0.56 mol) was added at 0° C. over period of 1 h. After complete addition the reaction mixture was stirred for 14 h at room temperature. The reaction mixture was cooled to 0° C., added cold water very slowly, and this mixture was diluted with aqueous saturated ammonium chloride (500 mL). The aqueous layer was extracted with ethyl acetate (500 mL) and washed with water (300 mL). The organic layer was concentrated under reduced pressure and the crude product was obtained (121 g) (R)-4-benzyloxymethyl-2,2-dimethyl-1,3-dioxolane (13) as a syrup. TLC (SiO₂) hexane/ethyl acetate (1:9) R_{f}˜0.26 (The crude material was subjected to next step without purification.) ¹H NMR (CDCl₃, 300 MHz): δ 1.39 (s, 3H, CH₃), 1.40 (s, 3H, CH₃), 3.41-3.55 (m, 2H), 3.68-3.74 (m, 1H), 4.01-4.05 (m, 1H), 4.20-4.31 (m, 1H), 4.55 (brs, 2H, OCH₂Ph), 7.25-7.35 (m, 5H, Ph-H).

### (S)-(−)-3-Benzyloxy-1,2-propanediol (14)

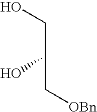

To a solution of (R)-4-benzyloxymethyl-2,2-dimethyl-1,3-dioxolane (13) (120 g) in methanol (700 mL) was added concentrated HCl (20 mL) and stirred at room temperature for 10 h. The reaction mixture was neutralized with sodium bicarbonate until the solution became neutral. The reaction mixture was filtered and concentrated under reduced pressure. The crude compound was dissolved in dichloromethane (600 mL) and organic layer was separated and dried over sodium sulfate. The organic layer was concentrated under reduced pressure. The crude compound was purified by column chromatography over a silica gel (230-400 mesh) with 10-20% ethyl acetate in hexane as eluent followed by 10% methanol in ethyl acetate to obtain (S)-(−)-3-benzyloxy-1,2-propanediol (14) (64 g, 93%) as syrup. TLC (SiO₂) ethyl acetate R_{f}˜0.44. ¹H NMR (CDCl₃, 300 MHz): δ 3.42-3.61 (m, 4H), 3.79-3.83 (m, 3H), 4.47 (s, 2H, OCH₂Ph), 7.23-7.32 (m, 5H, Ph-H).

### (S)-1,2-Bis-tetradecyloxy-3-O-benzylpropane (15)

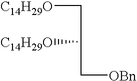

To a stirred suspension of sodium hydride (54.5 g, 1.36 mol, 60% in oil) in anhydrous DMF (220 mL) under argon atmosphere at 0° C., was added a solution of (S)-(−)-3-benzyloxy-1,2-propanediol (14) (62 g, 0.34 mol) in DMF (400 mL) over period of 1 h maintaining the internal temperature below 20° C. After stirring at room temperature for 2 h, tetradecyl bromide (377.4 g, 1.36 mol) was added at 0° C. over period of 2 h. After complete addition, the reaction mixture was stirred for 2 h at room temperature and the temperature was gradually increased to 70° C., then stirred for 5 h. The reaction mixture was cooled to 0° C., added cold water very slowly and diluted with aqueous saturated ammonium chloride (500 mL). The aqueous layer was extracted with ethyl acetate (1 L) and washed with water (3×1 L). The organic layer was concentrated under reduced pressure and the crude product was purified by column chromatography over a silica gel (70-230 mesh) eluting with 2-10% ethyl acetate in hexane to obtain (S)-1,2-bis-tetradecyloxy-3-O-benzylpropane (15) (146 g, 75%) as colorless oil. TLC (SiO₂) hexane/ethyl acetate (1:9) R_{f}˜0.53. ¹H NMR (CDCl₃, 300 MHz): δ 0.88 (t, J=6.7 Hz, 6H), 1.25 (brs, 44H), 1.49-1.58 (m, 4H), 3.39-3.60 (m, 9H), 4.54 (m, 2H, OCH₂Ph), 7.23-7.32 (m, 5H, Ph-H).

### (R)-1,2-Bis-tetradecyloxy-propan-3-ol (16)

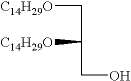

A solution of (S)-1,2-bis-tetradecyloxy-3-O-benzylpropane (15) (70 g, 0.12 mmol) was dissolved in ethyl acetate (280 mL) and hydrogenated with 10% palladium (3 g) for 12 h at a pressure of 50 psi. After filtration of the catalyst, the solution was evaporated under reduced pressure. The residue was dissolved in hot ethanol (500 mL) and kept at −20° C. overnight. The separated solid was filtered and dried under vacuum to obtain (R)-1,2-bis-tetradecyloxy-propan-3-ol (16) (54 g, 92%) as a white solid. TLC (SiO₂) hexane/ethyl acetate (1:9) R_{f}˜0.17. ¹H NMR (CDCl₃, 300 MHz): δ 0.88 (t, J=6.7 Hz, 6H), 1.25 (brs, 48H), 1.59-1.57 (m, 4H), 2.2 (t, J=5.7 Hz, 1H, OH), 3.37-3.73 (m, 9H).

### (S)-1,2-Bis-tetradecyloxy-3-bromopropane (17)

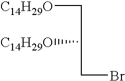

To a solution of (R)-1,2-bis-tetradecyloxy-propan-3-ol (16) (52 g, 0.1 mol) in anhydrous dichloromethane (280 mL) under argon atmosphere at 0° C. was added triphenylphosphine (35.1 g, 0.13 mol). A solution of carbon tetrabromide (46.2 g, 0.13 mol) in dichloromethane (240 mL) was added to the reaction mixture dropwise a period of 1 h. and further stirred at 0° C. for 3 h. The reaction mixture was diluted with water (500 mL) and the organic layer was separated, dried over sodium sulfate. The organic layer was concentrated under reduced pressure and the crude compound was purified by column chromatography over a silica gel (230-400 mesh) with 1-5% ethyl acetate in hexane to obtain (S)-1,2-bis-tetradecyloxy-3-bromopropane (17) (53 g, 90%) as colorless oil. TLC (SiO₂) hexane/ethyl acetate (1:9) R_{f}˜0.72. ¹H NMR (CDCl₃, 300 MHz): δ 0.88 (t, J=6.7 Hz, 6H), 1.25 (brs, 48H), 1.51-1.61 (m, 4H), 3.39-3.61 (m, 9H).

### (R)-1,2-Bis-tetradecyloxy-3-dimethylamino propane (18)

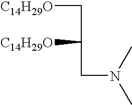

(S)-1,2-Bis-tetradecyloxy-3-bromopropane (17) (50 g, 0.09 mol) was dissolved in a 2M methanolic solution of dimethylamine (400 mL) in a screw-top pressure bottle. The pressure bottle was sealed and heated in an oil bath with stirring at 88-90° C. for 60 h. The pressure bottle was cooled and the solution was concentrated under reduced pressure. The crude residue was dissolved in ethyl acetate (500 mL) and washed with water (500 mL). The organic layer was concentrated under reduced pressure and purified by column chromatography over a silica gel (230-400 mesh) with 5-20% ethyl acetate in hexane as eluent to obtain (R)-1,2-bis-tetradecyloxy-3-dimethylamino propane (18) (41 g, 88%) as light colored oil. TLC (SiO₂) methanol/chloroform (1:9) R_{f}˜0.51. ¹H NMR (CDCl₃, 300 MHz): δ 0.88 (t, J=6.8 Hz, 6H), 1.25 (s, 44H), 1.51-1.58 (m, 4H), 2.25 (s, 6H, N—CH₃), 2.37 (t, J=4.6 Hz, 2H, N—CH₂), 3.41-3.62 (m, 7H).

### (R)-1,3-Bis-(1,2-ditetradecyloxypropyl-3-N,N-dimethyl-3-ethoxy ammonium bromide)propane-2-ol (19)

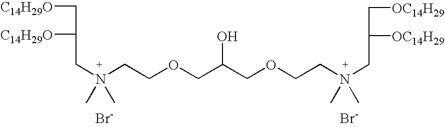

A solution of (R)-1,2-bis-tetradecyloxy-3-dimethylamino propane (18) (35.6 g, 69.8 mmol) and 1,3-bis-(2-bromoethoxy) propane-2-ol (10) (7.1 g, 23.2 mmol) in anhydrous ethanol (430 mL) was refluxed at 78-80° C. over a period of 5 days. The hot reaction mixture was transferred to erlenmeyer flask and added acetone (4.3 L) dropwise over period of 2 h. The mixture was keep at −20° C. over night. The solid was filtered and washed with cold acetone (500 mL) to obtain a colorless white solid (28 g). The crude solid was purified by recrystallization in the mixture of warm methanol (140 mL): acetone (1.4 L) and then stored at −20° C. overnight. The solid was separated, filtered and washed with cold acetone (300 mL). The recrystallization was repeated two times to get analytically pure sample. The compound was dried for 24 h and then over P₂O₅ for 36 h under vacuum to obtain (R)-cationic cardiolipin analog (19) (24 g, 78%) as a white solid. TLC (SiO₂) methanol/chloroform (1:9) R_{f}˜0.13. ¹H NMR(CDCl₃, 500 MHz): 60.88 (t, J=6.7 Hz, 12H), 1.25 (s, 88H), 1.52-1.71 (m, 8H), 3.41-3.68 (m, 30H), 3.95-4.19 (m, 13H), 4.63 (brs, 1H, OH). ¹³C NMR (CDCl₃, 125 MHz): δ 13.94, 22.52, 25.90, 26.04, 29.20, 29.28, 29.33, 29.44, 29.50, 29.52, 29.55, 29.88, 31.76, 52.99, 53.07, 53.49, 65.01, 65.07, 66.56, 68.66, 68.84, 69.17, 71.82, 72.62, 72.64, 73.30, 73.28. IR (cm⁻¹): 3409 (br, OH), 2918 (s), 2873 (s), 1468 (s), 1124 (br s). ESI-MS 1248.5 [M+1-Br], 584.2 [M+1-2Br/2] Mol. Formula C₇₃H₁₅₂Br₂N₂O₇; elemental analysis; calcd. C, 65.93; H, 11.52; N, 2.11; Br, 12.02; found C, 65.65; H, 11.49; N, 2.13; Br, 12.17.

### Example 4

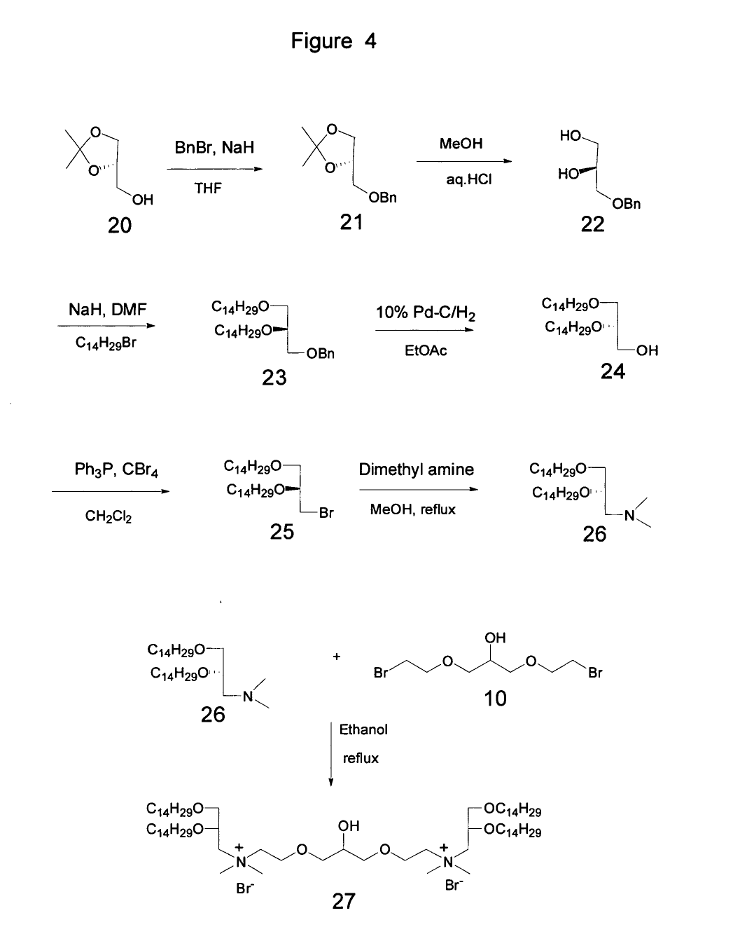

### Synthesis of Cationic Cardiolipin Analog (27) [FIG. 4]

### (S)-4-(Benzyloxymethyl)-2,2-dimethyl-1,3-dioxolane (21)

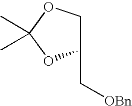

To a stirred suspension of sodium hydride (22.7 g, 0.57 mol, 60% in oil) in anhydrous terahydrofuran (350 mL) under argon atmosphere at 0° C., was added S(−)-2,2-dimethyl-1,3-dioxolane-4-methanol (20) (50 g, 0.37 mol) over period of 1 h maintaining the internal temperature below 20° C. After stirring at room temperature for 1 h, benzyl bromide (97.1 g, 0.56 mol) was added at 0° C. over period of 1 h. After complete addition the reaction mixture was stirred for 18 h at room temperature. The reaction mixture was cooled to 0° C., added few drops of ice water (20 mL) very slowly, and this mixture was diluted with aqueous saturated ammonium chloride (500 mL). The aqueous layer was extracted with ethyl acetate (750 mL). The organic layer was concentrated under reduced pressure and the crude product was obtained (83 g) of (S)-4-benzyloxymethyl-2,2-dimethyl-1,3-dioxolane (21) as a syrup. TLC (SiO₂) hexane/ethyl acetate (1:9) R_{f}˜0.26.

### (R)-(−)-3-Benzyloxy-1,2-propanediol (22)

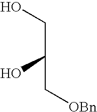

To a solution of (S)-4-(benzyloxymethyl)-2,2-dimethyl-1,3-dioxolane (21) (83 g) in methanol (800 mL) was added concentrated HCl (20 mL) and stirred at room temperature for 15 h. The reaction mixture was neutralized with sodium bicarbonate until the solution became neutral. The reaction mixture was filtered and concentrated under reduced pressure. The crude compound was dissolved in dichloromethane (200 mL) and organic layer was separated, dried over sodium sulfate and concentrated under reduced pressure. The crude compound was purified by column chromatography over a silica gel (230-400 mesh) with 10-20% ethyl acetate in hexane as eluent followed by 10% methanol in ethyl acetate to obtain (R)-(−)-3-benzyloxy-1,2-propanediol (22) (65 g, 94%) as syrup. TLC (SiO₂) ethyl acetate R_{f}˜0.44. ¹H NMR (CDCl₃, 300 MHz): δ 3.42-3.61 (m, 4H), 3.75-3.99 (m, 3H), 4.47 (s, 2H, OCH₂Ph), 7.22-7.32 (m, 5H, Ph-H).

### (R)-1,2-Bis-tetradecyloxy-3-O-benzylpropane (23)

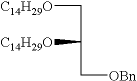

To a stirred suspension of sodium hydride (38.4 g, 0.96 mol, 60% in oil) in anhydrous DMF (200 mL) under argon atmosphere at 0° C., was added a solution of (R)-(−)-3-benzyloxy-1,2-propanediol (22) (35 g, 0.19 mol) in DMF (150 mL) over period of 1 h maintaining the internal temperature below 20° C. After stirring at room temperature for 2 h, tetradecyl bromide (266 g, 0.97 mol) was added at 0° C. over period of 2 h. After complete addition, the reaction mixture was stirred at room temperature for 2 h and the temperature was gradually increased to 60° C., then stirred for 20 h. The reaction mixture was cooled to 0° C., added few drops of ice water very slowly and diluted with aqueous saturated ammonium chloride (500 mL). The aqueous layer was extracted with hexane (3×500 mL) and washed with water (500 mL) and brine (500 mL). The organic layer was concentrated under reduced pressure and the crude product was purified by column chromatography over a silica gel (70-230 mesh) eluting with 2-10% ethyl acetate in hexane to obtain (R)-1,2-bis-tetradecyloxy-3-O-benzylpropane (23) (88 g, 80%) as a colorless oil. TLC (SiO₂) hexane/ethyl acetate (1:9) R_{f}˜0.53. ¹H NMR (CDCl₃, 300 MHz): δ 0.88 (t, J=6.6 Hz, 6H), 1.25 (brs, 44H), 1.49-1.58 (m, 4H), 3.39-3.60 (m, 9H), 4.54 (m, 2H, OCH₂Ph), 7.23-7.32 (m, 5H, Ph-H).

### (S)-1,2-Bis-tetradecyloxy-propan-3-ol (24)

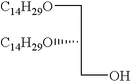

(R)-1,2-bis-tetradecyloxy-3-O-benzylpropane (23) (83.8 g, 0.14 mol) was dissolved in ethyl acetate (450 mL) and hydrogenated with 10% palladium on carbon (3.1 g) for 12 h at a pressure of 50 psi. After filtration of the catalyst, the solution was evaporated under reduced pressure. The residue was dissolved in hot hexane (200 mL) and kept at −20° C. overnight. The separated solid was filtered and dried to afford (S)-1,2-bis-tetradecyloxy-propan-3-ol (24) (64.8 g, 92%) as a white solid. TLC (SiO₂) hexane/ethyl acetate (1:9) R_{f}˜0.17. ¹H NMR (CDCl₃, 300 MHz): δ 0.88 (t, J=6.7 Hz, 6H), 1.25 (brs, 44H), 1.51-1.57 (m, 4H), 2.22 (t, J=5.2 Hz, 1H, OH), 3.40-3.73 (m, 9H).

### (R)-1,2-Bis-tetradecyloxy-3-bromopropane (25)

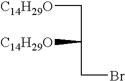

To a solution of (S)-1,2-bis-tetradecyloxy-propan-3-ol (24) (36.8 g, 59.0 mmol) in anhydrous dichloromethane (375 mL) under argon atmosphere at 0° C. was added triphenylphosphine (27.9 g, 106.0 mmol). A solution of carbon tetrabromide (36.2 g, 109.0 mmol) in dichloromethane (60 mL) added dropwise to reaction mixture over period of 1 h. The reaction mixture was further stirred at 0° C. for 2 h. and then diluted with water (3×300 mL). The organic layer was separated, dried over sodium sulfate, and concentrated under reduced pressure. The crude compound was purified by column chromatography over a silica gel (230-400 mesh) with 1-5% ethyl acetate in hexane to obtain (R)-1,2-bis-tetradecyloxy-3-bromopropane (25) (37.8 g, 87%) as colorless oil. TLC (SiO₂) hexane/ethyl acetate (1:9) R_{f}˜0.72. ¹H NMR (CDCl₃, 300 MHz): δ 0.88 (t, J=6.7 Hz, 6H), 1.25 (brs, 44H), 1.50-1.60 (m, 4H), 3.40-3.68 (m, 9H).

### (S)-1,2-Bis-tetradecyloxy-3-dimethylamino propane (26)

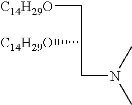

(R)-1,2-Bis-tetradecyloxy-3-bromopropane (25) (36.7 g, 0.07 mol) was dissolved in a 2M methanolic solution of dimethylamine (450 mL) in a screw-top pressure bottle. The pressure bottle was sealed and heated in an oil bath with stirring at 92° C. for 75 h. The pressure bottle was cooled and the solution was concentrated under reduced pressure. The crude residue was dissolved in ethyl acetate (500 mL) and washed with water (500 mL). The organic layer was concentrated under reduced pressure and purified by column chromatography over a silica gel (230-400 mesh) with 5-20% ethyl acetate in hexane as eluent to obtain (S)-1,2-bis-tetradecyloxy-3-dimethylamino propane (26) (28.7 g, 80%) as light colored oil. TLC (SiO₂) methanol/chloroform (2:8) R_{f}˜0.66. ¹H NMR (CDCl₃, 300 MHz): δ 0.88 (t, J=6.7 Hz, 6H), 1.25 (s, 44H), 1.51-1.57 (m, 4H), 2.15, 2.25 (s, 6H, N—CH₃), 2.34-2.36 (m, 2H, N—CH₂), 3.38-3.61 (m, 7H).

### (S)-1,3-Bis-(1,2-ditetradecyloxypropyl-3-N,N-dimethyl-3-ethoxy ammonium bromide)propane-2-ol (27)

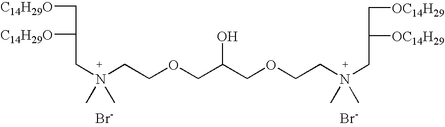

A solution of (S)-1,2-bis-tetradecyloxy-3-dimethylamino propane (26) (22.5 g, 43.9 mmol) and 1,3-bis-(2-bromoethoxy) propane-2-ol (10) (4.48 g, 14.6 mmol) in anhydrous ethanol (220 mL) was refluxed at 78-80° C. over a period of 5 days. The hot solution was transferred into a erlenmeyer flask and acetone (2.5 L) was added dropwise while stirring the solution. The flask was stored in the freezer (−25° C.) for 15 hrs. The white solid was filtered and washed with cold acetone (100 mL). The product was dissolved in chloroform (100 mL) and acetone (1 L) was added. The flask was stored at −25° C. for 15 hours. The separated white solid was filtered and washed with cold acetone (100 mL). The recrystallization procedure was repeated three times. The product was triturated with hexane (1 L) and filtered and dried over P₂O₅ under high vacuum for 24 h. to obtain (S)-cationic cardiolipin analog (27) (15.5 g, 80%) as a white soild. TLC (SiO₂) methanol/chloroform (1:9) R_{f}˜0.13. ¹H NMR (CDCl₃, 300 MHz): δ 0.88 (t, J=6.6 Hz, 12H), 1.25 (s, 88H), 1.52-1.71 (m, 8H), 3.40-3.71 (m, 36H), 3.92-4.11 (m, 7H), 4.74 (d, J=5.7 Hz, 1H, OH). ¹³C NMR (CDCl₃, 125 MHz): δ 13.91, 22.48, 25.86, 26.0, 29.16, 29.24, 29.30, 29.40, 29.46, 29.48, 29.51, 29.84, 31.72, 52.93, 53.01, 53.46, 64.98, 66.51, 86.63, 68.8, 69.12, 71.77, 72.61, 73.26. IR (cm⁻¹): 3397 (br, OH), 2917 (s), 1467 (s), 1122 (br s). ESI-MS 1248.5 [M+1-Br], 584.4 [M+1-2Br/2].

### Example 5

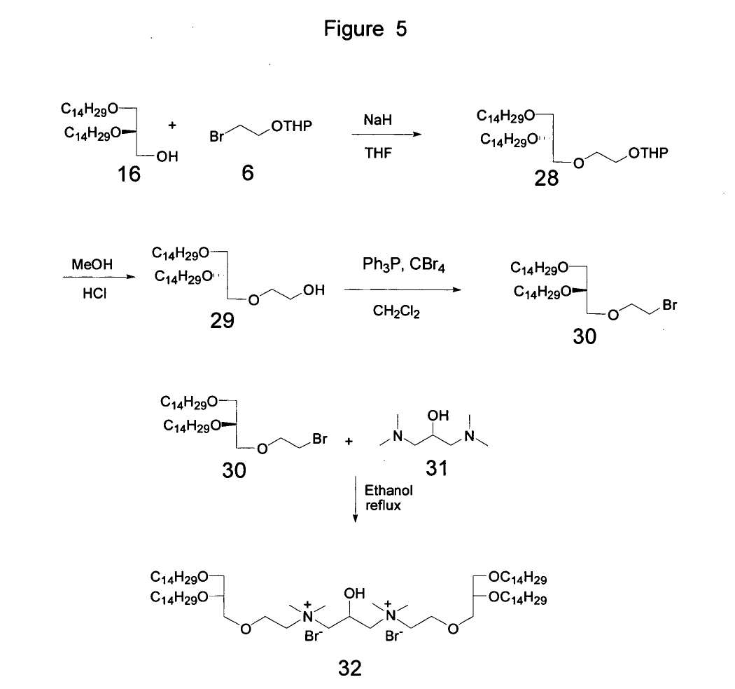

### Synthesis of Cationic Cardiolipin Analog (32) [FIG. 5]

### 2-[2-(2,3-Bis-tetradecyloxy-propoxy)-ethoxy]-tetrahydro-pyran (28)

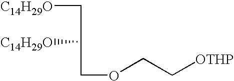

To a stirred suspension of sodium hydride (3.3 g, 82.6 mmol, 60% in oil) in anhydrous tetrahydrofuran (50 mL) under argon atmosphere at 0° C., a solution of 1,2-bis (tetradecyloxy) propane-3-ol (16) (20 g, 41.3 mmol) in THF (150 mL) was added over a period of 2 h maintaining the temperature below 15° C. After stirring at room temperature for 2 h, 2-(2-bromoethoxy)tetrahydro-2H-pyran (6) (25.9 g, 123 mmol) was added at 0° C. over a period of 3 h maintaining the temperature below 110° C. The reaction mixture was stirred at room temperature for 48 h. The reaction mixture was cooled to 0° C., ice water was added very slowly to quench excess sodium hydride and diluted with aqueous saturated ammonium chloride (300 mL) and extracted with ethyl acetate (2×150 ml). The organic layer was washed with water (100 mL), dried over sodium sulfate and concentrated under reduced pressure. The crude compound was purified by column chromatography over a silica gel (230-400 mesh) with 1-8% ethyl acetate in hexane to obtain 2-[2-(2,3-bis-tetradecyloxypropoxy) ethoxy]tetrahydropyran (28) (16.3 g, 66%) as colorless oil. TLC (SiO₂) hexane:ethyl acetate (1:4) R_{f}˜0.30 ¹H NMR (CDCl₃, 300 MHz): δ 0.88 (t, J=6.7 Hz, 12H), 1.25 (s, 44H), 1.53-1.87 (m, 10H), 3.40-4.04 (m, 15H), 4.62-4.67 (m, 1H).

### 2-(2,3-Bis-tetradecyloxy-propoxy)-ethanol (29)

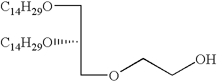

To a solution of 2-[2-(2,3-bis-tetradecyloxypropoxy) ethoxy]tetrahydropyran (28) (16 g, 26.75 mmol) in methanol (500 mL) was added 1N HCl in ether (5 mL) and stirred at room temperature for 2 h. The reaction mixture was neutralized with sodium bicarbonate until the solution became neutral. The reaction mixture was filtered and concentrated under reduced pressure. The residue was dissolved in ethyl acetate (500 mL) and washed with water (200 mL) and dried over sodium sulfate. The organic layer was concentrated under reduced pressure and crude compound was purified by column chromatography over a silica gel (230-400 mesh) eluting with 10-15% ethyl acetate in hexane to obtain 2-(2,3-bis-tetradecyloxypropoxy) ethanol (29) (10 g, 71%) as colorless oil. TLC (SiO₂) hexane/ethyl acetate (1:4) R_{f}˜0.18 ¹H NMR (CDCl₃, 300 MHz): δ 0.88 (t, J=6.7 Hz, 12H), 1.25 (s, 44H), 1.51-161 (m, 4H), 2.58 (t, J=6 Hz, OH), 3.41-3.71 (m, 13H).

### 1-[1-(2-Bromo-ethoxymethyl]-2-tetradecyloxyethoxyl tetradecane (30)

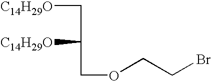

To a solution of 2-(2,3-bis-tetradecyloxypropoxy)ethanol (29) (20 g, 37.8 mmol) in anhydrous dichloromethane (200 mL) under argon atmosphere at 0° C. was added triphenylphosphine (14.8 g, 56.8 mmol) followed by carbon tetrabromide (17.5 g, 53.0 mmol). The reaction mixture was stirred at 0° C. for 2 h. The reaction mixture was diluted with water (100 mL) and the organic layer was separated, dried over sodium sulfate. The organic layer was concentrated under reduced pressure and the crude compound was purified by column chromatography over a silica gel (230-400 mesh) with 5% ethyl acetate in hexane to obtain 1-[1-(2-bromoethoxymethyl)-2-tetradecyloxyethoxy] tetradecane (30) (17 g, 76%) as colorless oil. TLC (SiO₂) hexane/ethyl acetate (1:5) R_{f}˜0.65. ¹H NMR (CDCl₃, 300 MHz): δ 0.88 (t, J=6.7 Hz, 12H), 1.25 (s, 44H), 1.51-157 (m, 4H), 3.40-3.61 (m, 11H), 3.79 (t, J=6 Hz, 2H).

### 1,3-Bis-(1,2-ditetradecyloxy-4-oxa-hexyl-6-N,N-dimethyl ammonium bromide)propane-2-ol (32)

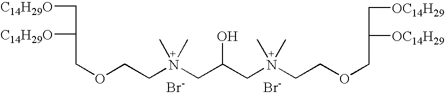

A solution of 1-[1-(2-bromoethoxymethyl)-2-tetradecyloxyethoxy]-tetradecane (30) (6 g, 10.15 mmol) and 1,3-bis (dimethylamino)-2-propanol (31) (0.48 g, 3.38 mmol) in anhydrous ethanol (65 mL) was refluxed at 78-80° C. over a period of 5 days. The hot solution was transferred into a erlenmeyer flask and acetone (650 mL) was added dropwise while stirring the solution. The flask was stored in the freezer (−25° C.) for overnight. The white solid was filtered and washed with cold acetone (100 mL). The solid was dissolved in dichloromethane and acetone was added (ratio 1:10). The flask was stored at −25° C. for overnight. The white solid was filtered and washed with cold acetone (20 mL). The recrystallization procedure was repeated two times. The product was dried under vacuum to obtain cationic cardiolipin analog (32) (0.82 g, 18%) as a white soild. TLC (SiO₂) methanol/chloroform (1:9) R_{f}˜0.12 ¹H NMR δ (CDCl₃, 300 MHz): δ 0.88 (t, J=6.8 Hz, 6H), 1.25 (s, 88H), 1.49-1.60 (m, 8H), 3.39-3.82 (m, 31H), 3.81-4.01 (m, 8H), 4.52 (d, J=12.6 Hz, 2H), 5.21-5.35 (m, 1H). ESI-MS: 1248.7 [M+1-Br⁻], 584.6 [M+1-2Br⁻/2]. Mol. Formula C₇₃H₁₅₂Br₂N₂O₇; elemental analysis; calcd. C, 65.93; H, 11.52; N, 2.11; Br, 12.02; found C, 64.46; H, 11.34; N, 2.21, Br, 11.80.

### Example 6

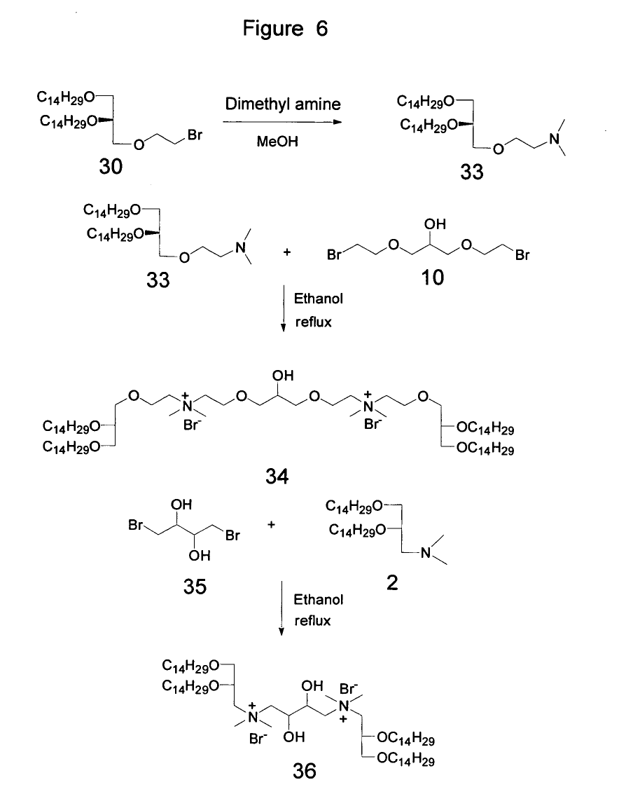

### Synthesis of Cationic Cardiolipin Analog (34) [FIG. 6]

### [2-(2,3-Bis-tetradecyloxypropoxy) ethyl]-dimethylamine (33)

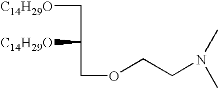

1-[1-(2-Bromoethoxymethyl)-2-tetradecyloxyethoxy] tetradecane (30) (10 g, 16.92 mmol) was in a 2M methanolic solution of dimethylamine (100 mL) in a screw-top pressure bottle. The pressue bottle was sealed and heated in an oil bath with stirring at 88-90° C. for 60 h. The pressure bottle was cooled and the solution was concentrated under reduced pressure. The crude compound was dissolved in ethyl acetate (300 mL) and washed with water (100 mL). The organic layer was concentrated under reduced pressure and purified by column chromatography over a silica gel (230-400 mesh) with 5-20% ethyl acetate in hexane as eluent to obtain [2-(2,3-bis-tetradecyloxypropoxy) ethyl] dimethylamine (33) (8 g, 85%) as light colored oil. TLC (SiO₂) methanol/chloroform (1:9) R_{f}˜0.46. ¹H NMR (CDCl₃, 300 MHz): δ 0.88 (t, J=6.7 Hz, 12H), 1.25 (s, 44H), 1.52-1.56 (m, 4H), 2.17 (s, 6H, N—CH₃), 2.78 (t, J=5.8 Hz, 2H), 3.39-3.46 (m, 4H), 3.49-3.56 (m, 5H), 3.71 (t, J=5.4 Hz, 2H)

### 1,3-Bis-(1,2-ditetradecyloxy-4,10-dioxa-decyl-7-N,N-dimethyl ammonium bromide)propane-2-ol (34)

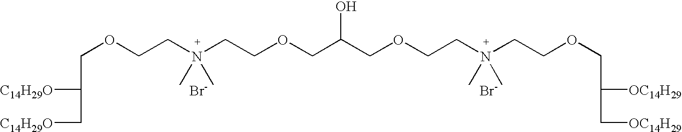

A solution of (R)-[2-(2,3-bis-tetradecyloxypropoxy)ethyl] dimethylamine (33) (5 g, 8.99 mmol) and 1,3-bis-(2-bromoethoxy)propane-2-ol (10) (0.91 g, 2.99 mmol) in anhydrous ethanol (60 mL) was refluxed at 78-80° C. over a period of 5 days. The hot reaction mixture was transferred to a erlenmeyer flask and added acetone (600 mL) dropwise over a period of 2 h and keep at −20° C. over night. The solid was filtered and washed with cold acetone (100 mL) to obtain a colorless white solid (5 g). The crude solid was purified by recrystallization in warm methanol: acetone (ratio of 1:10) and then stored at −20° C. overnight. The solid was separated, filtered and washed with cold acetone (50 mL). The recrystallization was repeated two times to get pure compound. The compound was dried under vacuum to obtain cationic cardiolipin analog (34) (1.82 g, 43%) as a white solid. TLC (SiO₂) methanol/chloroform (1:9) R_{f}˜0.13. ¹H NMR (CDCl₃, 300 MHz): δ 0.88 (t, J=6.9 Hz, 12H), 1.25 (s, 88H), 1.48-1.59 (m, 8H), 3.38-3.65 (m, 35H), 3.89-4.13 (m, 16H), 4.77 (brs, 1H, OH). ESI-MS: 1336.4 [M+1-Br⁻], 628.4 [M+1-2Br⁻/2]. Mol. Formula C₇₇H₁₆₀Br₂N₂O₉; elemental analysis; calcd. C, 65.22; H, 11.37; N, 1.98; Br, 11.27; found C, 63.94; H, 11.28; N, 2.03; Br, 9.94.

### Example 7

### Synthesis of Cationic Cardiolipin Variant Analog (36) [FIG. 6]

### (R,S)-1,3-Bis-(1,2-ditetradecyloxypropyl-3-N,N-dimethyl ammonium bromide)butane-2,3-diol (36)

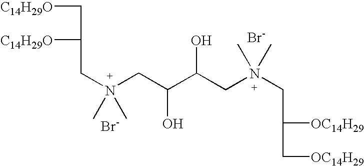

A solution of 1,2-bis-(tetradecyloxy)-3-dimethylamino propane (2) (6 g, 11.72 mmol) and 1,4-dibromo-2,3-butanediol (35) (1.16 g, 4.68 mmol) in anhydrous ethanol (72 mL) was refluxed for a period of 7 days. The reaction mixture was cooled and the solvent was evaporated to give a crude waxy solid. The crude compound was dissolved in hexane (200 mL) and stirred at room temperature for 6 h, kept at 0° C. overnight. The separated solid was filtered and washed with hexane (8×10 mL) to remove the starting material 1,2-bis-(tetradecyloxy)-3-dimethylamino propane. The crude material was subjected to column chromatography (silica-gel, 70-230 mesh,) eluting with 1-10% methanol in dichloromethane to obtain cationic cardiolipin varient analog (36) (1.9 g, 32% 0 as a white solid. TLC (SiO₂) methanol/chloroform (1:9) R_{f}˜0.07 ¹H NMR (CDCl₃, 500 MHz): δ 0.88 (t, J=6.8 Hz, 12H), 1.24-1.31 (m, 88H), 1.51-1.62 (m, 8H), 2.13 (brs, 2H, OH), 3.33-3.65 (m, 22H), 3.75-3.82 (m, 6H), 4.05-4.25 (m, 6H), 4.68 (brs, 2H), 4.39 (d, J=13 Hz, 2H. ¹³C NMR (CDCl₃, 125 MHz): δ 14.05, 22.63, 25.98, 26.14, 29.30, 29.42, 29.63, 31.87, 52.58, 53.28, 54.83, 67.27, 68.87, 69.40, 72.04, 73.17. ESI-MS: 1190.3 [M+1-Br⁻], 555.3 [M+1-2Br⁻/2]. Mol. Formula C₇₁H₁₄₈Br₂N₂O₆; Elemental analysis; calcd. C, 66.32; H, 11.60; N, 2.18; Br, 12.43; found C, 65.20; H, 11.46; N, 2.19; Br, 12.24.

### Example 8

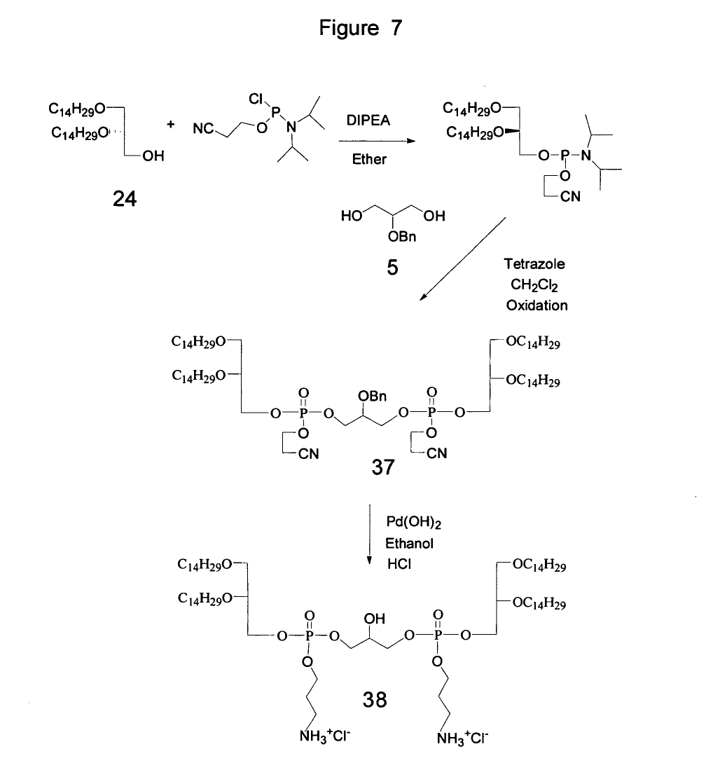

### Synthesis of Cationic Cardiolipin Variant Analog (38) [FIG. 7]

### 2-Benzyl-1,3-bis [(1,2-dimyristoyl-sn-glycero-3)-phosphoryl]glycerol dicyanoethyl ester compound (37) Prepared Following the Procedure Described in U.S. patent application (Ahmad et al. PCT/US03/27806)

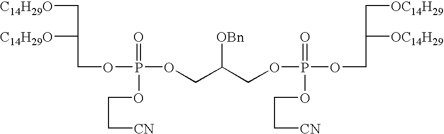

To a mixture of 1,2-myristyl-sn-glycerol (24) (9.2 g, 19.01 mmol) and N, N-diisopropylethylamine (5.46 g, 42.26 mmol) in anhydrous ether (150 mL) under argon atmosphere was added 2-cyanoethyl diisopropylchlorophosphoramidite (5 g, 21.13 mmol). The mixture was stirred at room temperature for 3 h, the diisopropylamine hydrochloride was filtered, and the filtrate was concentrated under reduced pressure and crude material was dried under vacuum for 1 h to gave phosphoramidite intermediate (13 g) as such used for the next phosphorylation.

To a mixture of above phosphoramidite and 1H-tetrazole (1.59 g, 22.77 mmol, 0.45 M solution in acetonotrile) in anhydrous CH₂Cl₂ (80 mL) was added a solution of 2-benzyloxy 1,3-propanediol (5) (1.55 g, 8.54 mmol) in CH₂Cl₂ (20 mL). The reaction mixture was stirred at room temperature for 3 h and cooled to −40° C. and t-butylhydroperoxide (6.8 g, 75.91 mmol) was added in portions. After stirring at −40° C. for 30 minutes, the reaction mixture was warmed to room temperature, diluted with CH₂Cl₂ (200 mL), washed with 5% aqueous NaHCO₃ (50 mL), brine (50 mL) and dried over sodium sulfate. The organic layer was concentrated under reduced pressure. The residue was purified on silica-gel (230-400 mesh) eluting with 50-75% ethyl acetate in hexane to give (37) (7.1 g, 61%) as colorless syrup. TLC (SiO₂) ethyl acetate/hexane (3:1) R_{f}˜0.29 ¹H NMR (CDCl₃, 300 MHz): δ 0.88 (t, J=7.0 Hz, 12H), 1.22-1.39 (m, 88H), 1.51-1.57 (m, 8H), 2.58-2.79 (m, 4H), 3.39-3.87 (m, 13H), 4.04-4.32 (m, 10H), 4.59-4.68 (m, 2H), 7.27-7.36 (m, 5H).

### 1,3-Bis [(1,2-ditetradecyloxy-sn-glycero-3)-phosphoryl]glycerol diammonium chloride-n-propane ester (38)

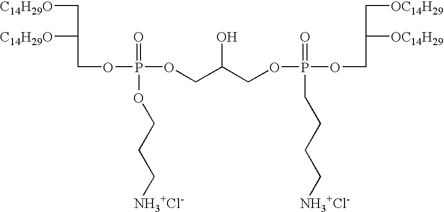

A solution of 2-benzyl-1,3-bis-[(1,2-dimyristyl-sn-glycero-3)-phosphoryl]glycerol dicyanoethyl ester (37) (0.74 g, 0.53 mmol) in ethanol (25 mL) was hydrogenated at 50 psi over Pd (OH)₂ (210 mg) for 24 h. The catalyst was filtered through celite bed and washed with ethanol. The ethanol layer was concentrated under reduced pressure and dried under vacuum for overnight to give cationic cardiolipin variant analog (38) 0.6 g a as syrup. ¹H NMR (CDCl₃, 300 MHz): δ 0.88 (t, J=6.8 Hz), 1.24 (brs, 88H), 1.51-1.61 (m, 8H), 2.80-2.88 (m, 1H), 3.01-3.3.18 (m, 3H), 3.40-4.21 (m, 28H). The amino compound was converted to hydrochloride salt by adding 1N HCl solution in ether.

### Example 9

### Synthesis of Cationic Cardiolipin Ester Analog (44) FIG. 81

### Toluene-4-sulfonic acid 2,2-dimethyl-[1,3]dioxolan-4-ylmethyl ester (39)

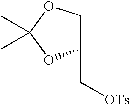

To a mixture of R (−)-2,2-dimethyl-1,3-dioxolane-4-methanol (12) (40 g, 0.30 mol) and pyridine (250 mL) at 0° C. was added tosyl chloride (63.3 g, 0.33 mol) portionwise over a period of 1 h, then allow to warm up to room temperature and stirred for 12 h. The reaction mixture was concentrated under reduced pressure and crude compound was diluted with water (1.5 L) and extracted with ethyl acetate (500 mL). The organic layer was dried over sodium sulfate and concentrated under reduced pressure to obtain toluene-4-sulfonic acid 2-[2-(2-methoxyethoxy) ethoxy] ethyl ester (39) (87 g) as light colored solid. TLC (SiO₂) ethyl acetate/hexane (1:4) R_{f}˜0.22. ¹H NMR (CDCl₃, 300 MHz): δ 1.29 (s, 3H), 1.32 (s, 3H), 2.44 (s, 3H, Ar—CH₃), 2.73 (dd, J=10.6 Hz, 1H), 3.97-4.0 (m, 3H), 4.22-4.30 (m, 1H), 7.35 (dd, J=8.4 Hz, 2H, Ar—H), 7.77 (dd, J=8.4 Hz, 2H, Ar—H) 2,2-Dimethyl-[1,3]dioxolan-4-ylmethyl dimethylamine (41).

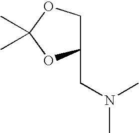

Method 1: To a mixture of (R)-(+)-2,2-dimethyl-1,3-dioxolane-4-carboxaldehyde (40) (31 g, 0.23 mol) and 2M methanolic solution of dimethylamine (70 mL) in anhydrous methanol (100 mL) under argon atmosphere was added anhydrous sodium sulfate (50 g). The mixture was stirred at room temperature for 2 h. The reaction was cooled to 0° C. and sodium borohydride (7.1 g, 0.18 mol) was added in portions and stirred at room temperature overnight. The reaction mixture was concentrated under reduced pressure. The crude compound was dissolved in ethyl acetate (100 mL) and washed with water (100 mL). The organic layer was concentrated under reduced pressure and purified by column chromatography over a silica gel (230-400 mesh) with 20-40% ethyl acetate in hexane as eluent to obtain 2,2-dimethyl-[1,3]dioxolan-4-ylmethyl dimethylamine (41) (7 g, 19%) as light colored oil. TLC (SiO₂) methanol/chloroform (1:9) R_{f}−0.45. ¹H NMR (CDCl₃, 500 MHz): δ 1.35 (s, 3H), 1.41 (s, 3H), 2.28 (s, 6H, N—CH₃), 2.35 (dd, J=12.5 Hz, N—CH₂), 2.49 (dd, J=12.5 Hz, N—CH₂), 3.58 (dd, J=8 Hz, 1H), 4.07 (dd, J=8 Hz, 1H), 4.20-4.25 (m, 1H).

Method 2: Toluene-4-sulfonic acid 2-[2-(2-methoxyethoxy) ethoxy] ethyl ester (39) (50 g, 0.17 mol) was dissolved in a 2M methanolic solution of dimethylamine (400 mL) in a screw-top pressure bottle. The pressure bottle was heated in an oil bath with stirring at 88-90° C. for 48 h. The pressure bottle was cooled and the solution was concentrated under reduced pressure. The crude compound was dissolved in ethyl acetate (100 mL) and washed with water (100 mL). The organic layer was concentrated under reduced pressure to obtain 2,2-dimethyl [1,3]dioxolan-4-yl-methyl dimethylamine (41) (11 g, 40%) as light colored oil.

### 3-Dimethylaminopropane-1,2-diol (42)

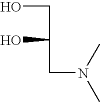

To a solution of 2,2-dimethyl-[1,3]dioxolan-4-yl-methyl dimethylamine (41) (5 g, 31.44 mmol) in methanol (100 mL) was added 1M HCl in ether (10 mL) and stirred at room temperature for 7 h. The reaction mixture was neutralized with sodium bicarbonate until the solution became neutral. The reaction mixture was filtered and concentrated under reduced pressure. The crude compound was extracted with warm ethyl acetate (5×100 mL). The organic layer was concentrated under reduced pressure to obtain 3-dimethylamino propane-1,2-diol (42) (1.4 g, 37%) as light colored syrup. TLC (SiO₂) methanol:chloroform (1:4) R_{f}˜0.13. ¹H NMR (CDCl₃, 300 MHz): δ 2.23 (dd, J=5.1 Hz, 1H, N—CH₂), 2.38 (s, 6H, N—CH₃), 2.47-2.56 (m, 1H, N—CH₂), 3.49 (dd, J=11.2 Hz, 1H), 3.68-3.81 (m, 4H).

### 1,2-Dimyristoyl-3-dimethylamino propane (43)

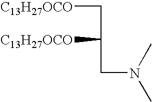

To an ice cooled solution of (R)-(+)-3-dimethylaminopropane-1,2-diol (42) (3 g, 25.2 mmol) and pyridine (100 mL) was added myristoyl chloride (19.8 g, 75.6 mmol) dropwise over a period of 30 minutes. The reaction mixture was stirred at room temperature for 4 days and concentrated under reduced pressure. The crude compound was diluted with CH₂Cl₂ (200 mL), washed with water (100 mL) and brine (100 mL) and dried over sodium sulfate. The organic layer was concentrated under reduced pressure and residue was purified on silica-gel (230-400 mesh) with 5% EtOAc in hexane to gave (43) (2.9 g, 21%) as a light colored syrup. TLC (SiO₂) ethyl acetate R_{f}˜0.54. ¹H NMR (CDCl₃, 300 MHz): δ 0.88 (t, J=6.9 Hz, 6H), 1.25 (brs, 40H, -), 1.60 (t, J=6.9 Hz, 4H), 2.13 (brs, 2H, OH), 3.33-3.65 (m, 22H), 3.75-3.82 (m, 6H), 2.25 (s, 6H, N—CH₃), 2.30-2.33 (m, 1H, N—CH₂), 2.44 (t, J=6.3 Hz, 1H, N—CH₂), 4.08 (dd, J=11.8 Hz, 1H), 4.36 (dd, J=11.8 Hz, 1H), 5.15-5.23 (m, 1H).

### (R)-1,3-Bis-(1,2-dimyristoylpropyl-3-N,N-dimethyl-3-ethoxy ammonium bromide)propane-2-ol (44)

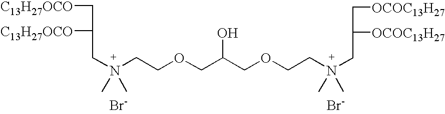

A solution of 1,2-bis-(tetradecyloxy)-3-dimethylamine (43) (4 g, 7.42 mmol) and 1,3-bis-(2-bromoethoxy) propane-2-ol (10) (0.75 g, 2.47 mmol) in anhydrous ethanol (45 mL) were refluxed for a period of 5 days. The reaction mixture was cooled and the solvent was evaporated to give a crude waxy solid. The compound was subjected to column chromatography (silica-gel, 70-230 mesh,) eluting with 0-10% methanol in dichloromethane to obtain cationic cardiolipin ester analog (44) (67 mg, 2%). TLC (SiO₂) methanol/chloroform (1:9) R_{f}˜0.10 ¹H NMR (CDCl₃, 300 MHz): δ 0.88 (t, J=7.2 Hz), 1.25 (brs, 80H), 1.60 (brs, 8H), 2.37 (t, J=7.5 Hz, 8H), 3.48-3.83 (m, 24H), 3.99-4.20 (m, 8H), 4.65-4.75 (m, 2H), 5.14-5.25 (m, 2H).

All references, including publications, patent applications, and patents, cited herein, including, but not limited to the following list, are hereby incorporated by reference to the same extent as if each reference were individually and specifically indicated to be incorporated by reference and were set forth in its entirety herein.

### REFERENCES

1). Ahmad, M. U.; Ukkalam, M. K.; Ahmad, I. International Patent Application PCT/US03/27806.
2). Bhattacharya, S.; De, S. Chem. Eur. J. 1999, 5(8), 2335-47.
3). Felgner, P. L.; Gadek, T. R.; Holm, M.; Roman, R.; Chan, H. W.; Wenz, M.; Northrop, J. P.; Ringold, G. M.; Danielsen, M. Proc Natl Acad Sci USA 1987, 84, 7413-7417
4). Felgner, P. L.; Fe, R. S.; Kumar, R.; Basava, C.; Border, R. C., Felgner-H, J-Y. PCT/US/1993/5,264,618
5). Felgner, P. L.; Fe, R. S.; Kumar, R.; Basava, C.; Border, R. C., Felgner-H, J-Y. PCT/US/1995/5,459,127
6). Grunner, S. M.; Jain, M. H. Biochem. Biophys. Acta. 1985, 352-355
7). Hostetler, K. Y.; Kumar, R.; Stuhmiller, L. M. PCT/US/1993/5,223,263.
8). Hostetler, K. Y.; Kumar, R.; Sridhar, N. C. PCT/US/1998/5,827,831.
9). Hostetler, K. Y.; Kumar, R.; Stuhmiller, L. M. PCT/US/2002/6,448,392.
10). Kunkel, L. M.; Hoffman, E. P. Brit. Med. Bull. 1989, 45(3), 630-43.
11). Miller, A. Angew. Chem. Int. Ed. 1998, 37,1768-85
12). Tryell, A.; Heath, T. D.; Colley, C. M.; Ryman, B. E. Biochem. Biophys Acta. 1976, 457, 259-302.
13). Ts'O, P. O.; Miller, L.; Aurelian, L.; Murakami, A.; Agris, C.; Blake, K. R.; Lin, S. B.; Lee, B. L.; Smith, C. C. Annals New York Acad. Sci. 1987, 507, 220-241
14). Wheeler, C. J. PCT/WO/2000/73263 A1
15). Wheeler, C. J.; Sukhu, L.; Yang, G.; Tsai, Y.; Bustamente, C.; Felgner, P.; Norman, J.; Manthorpe, M. Biochem. Biophys. Acta. 1996, 1280, 1-11.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments might become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A cationic cardiolipin analog having the general structure I. 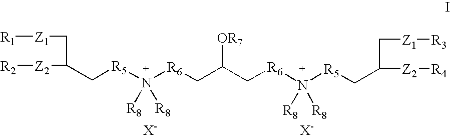
wherein Z₁ and Z₂ are the same or different and are —O—C(O)—, —O—, —S—, or —NH—C(O)—;
R₁, R₂, R₃, and R₄ are the same or different and are H, C₁ to C₃₂ saturated or unsaturated alkyl, alkenyl, or alkynyl, groups, optionally hydroxylated, aminenated, thiolated, epoxylated, cyclolated, PEGylated, halogenated, or substituted with combinations thereof;
R₅ and R₆ are the same or different and can be either absent or comprise a linker comprising a C₁-C₃₂ alkyl, substituted alkyl, cycloalkyl, substituted cycloalky, or an alkyloxy or substituted alkyloxy group such as a PEGylated ether containing from 1 to 500 PEG (polyethylene glycol) units;
R₇ is hydrogen, alkyl, substituted alkyl, alkyloxy, substituted alkyloxy, cycloalkyl, substituted cycloalkyl, alkenyl, alkynyl, alkanoyl, alkenoyl, alkynoyl, optionally hydroxylated, aminenated, thiolated, epoxylated, cyclolated, PEGylated, halogenated, or substituted with combinations thereof; or an alkyloxy or substituted alkyloxy group such as PEGylated ether containing from 1 to 500 PEG (polyethylene glycol) units;
an amino acid, folic acid, a peptide, a peptidomimetic moiety, a dipeptide, a polypeptide, a protein, a carbohydrate, a saccharide or polysaccharide, a polyamine, a heterocyclic, a nucleoside, or a polynucleotide;
the R₈ groups are the same or different and are C₁ to C₂₅ saturated or unsaturated alkyl, alkyloxy, substituted alkyl, or substituted alkyloxy;
X is a non-toxic anion.

2. The analog of claim 1, wherein the analog is a cationic cardiolipin ether having the structure of formula II: 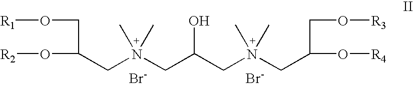
wherein R₁, R₂, R₃, and R₄ are the same or different and are H, or C₁ to C₃₂ alkyl, alkenyl, or alkynyl.

3. The analog of claim 1, wherein the analog is a cationic cardiolipin ester having the structure of formula III: 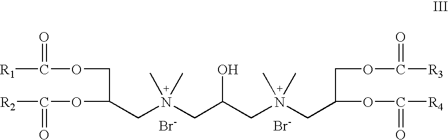
wherein R₁, R₂, R₃, and R₄ are the same or different and are H, or C₁ to C₃₁, alkyl, alkenyl, or alkynyl.

4. The analog of claim 1, wherein the analog is a cationic cardiolipin ether having the structure of formula IV: 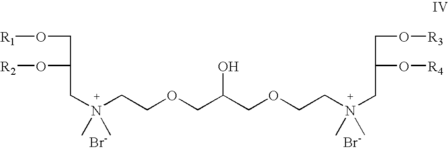
wherein R₁, R₂, R₃, and R₄ are the same or different and are H, or C₁ to C₃₂ alkyl, alkenyl, or alkynyl.

5. The analog of claim 1, wherein the analog is a cationic cardiolipin ester having the structure of formula V: 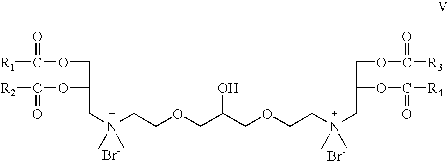
wherein R₁, R₂, R₃, and R₄ are the same or different and are H, or C₁ to C₃₁ alkyl, alkenyl, or alkynyl.

6. The analog of claim 1, wherein the analog is a cationic cardiolipin ether having the structure of formula VI: 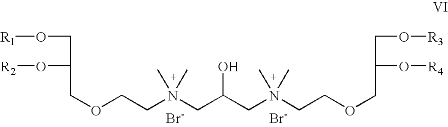
wherein R₁, R₂, R₃, and R₄ are the same or different and are H, or C₁ to C₃₂ alkyl, alkenyl, or alkynyl.

7. The analog of claim 1, wherein the analog is a cationic cardiolipin ester having the structure of formula VII: 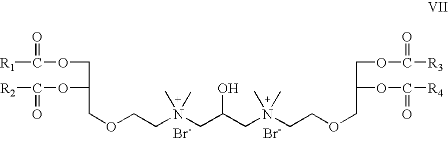
wherein R₁, R₂, R₃, and R₄ are the same or different and are H, or C₁ to C₃₁ alkyl, alkenyl, or alkynyl.

8. The analog of claim 1, wherein the analog is a cationic cardiolipin ether having the structure of formula VIII: 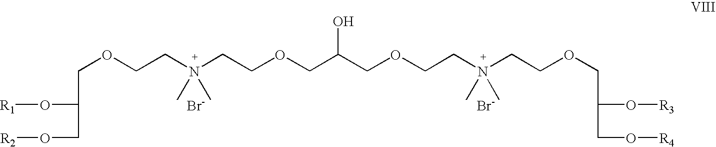
wherein R₁, R₂, R₃, and R₄ are the same or different and are H, or C₁ to C₃₂ alkyl, alkenyl, or alkynyl.

9. The analog of claim 1, wherein the analog is a cationic cardiolipin ester having the structure of formula IX: 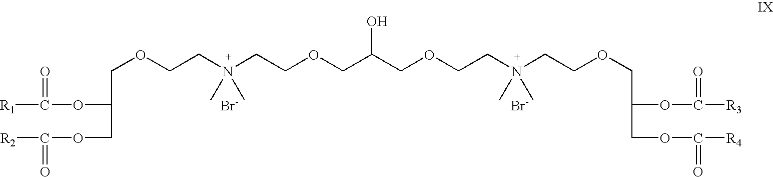
wherein R₁, R₂, R₃, and R₄ are the same or different and are H, or C₁ to C₃₁, alkyl, alkenyl, or alkynyl.

10. A cationic cardiolipin analog having the general structure X. 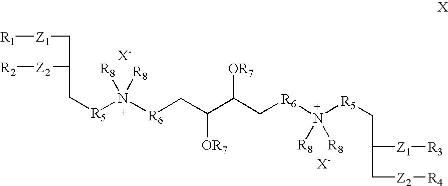
wherein Z₁ and Z₂ are the same or different and are —O—C(O)—, —O—, —S—, or —NH—C(O)—;
R₁, R₂, R₃, and R₄ are the same or different and are H, C₁ to C₃₂ saturated or unsaturated alkyl, alkenyl, or alkynyl, groups, optionally hydroxylated, aminenated, thiolated, epoxylated, cyclolated, PEGylated, halogenated, or substituted with combinations thereof;
R₅ and R₆ are the same or different and can be either absent or comprise a linker comprising a C₁-C₃₂ alkyl, substituted alkyl, cycloalkyl, substituted cycloalky, or an alkyloxy or substituted alkyloxy group such as PEGylated ether containing from 1 to 500 PEG (polyethylene glycol) units;
the R₇ groups can be the same or different and are hydrogen, alkyl, substituted alkyl, alkyloxy, substituted alkyloxy, cycloalkyl, substituted cycloalkyl, alkenyl, alkynyl, alkanoyl, alkenoyl, alkynoyl, optionally hydroxylated, aminenated, thiolated, epoxylated, cyclolated, PEGylated, halogenated, or substituted with combinations thereof; or an alkyloxy or substituted alkyloxy group such as PEGylated ether containing from 1 to 500 PEG (polyethylene glycol) units; an amino acid, a peptide, a peptidomimetic moiety, a dipeptide, a polypeptide, a protein, a carbohydrate, a saccharide or polysaccharide, a polyamine, a heterocyclic, a nucleoside, folic acid or a polynucleotide;
the R₈ groups are the same or different and are C₁ to C₂₅ saturated or unsaturated alkyl, alkyloxy, substituted alkyl, or substituted alkyloxy;
X is a non-toxic anion.

11. The analog of claim 10, wherein the analog is a cationic cardiolipin ether having the structure of formula XI: 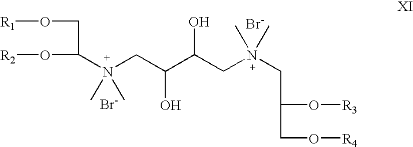
wherein R₁, R₂, R₃, and R₄ are the same or different and are H, or C₁ to C₃₂ alkyl, alkenyl, alkynyl.

12. The analog of claim 10, wherein the analog is a cationic cardiolipin ester having the structure of formula XII: 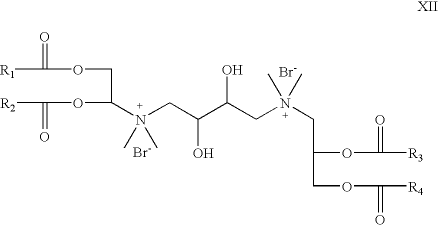
wherein R₁, R₂, R₃, and R₄ are the same or different and are H, or C₁ to C₃₁ alkyl, alkenyl, or alkynyl.

13. A cationic cardiolipin analog having the general structure XIII. 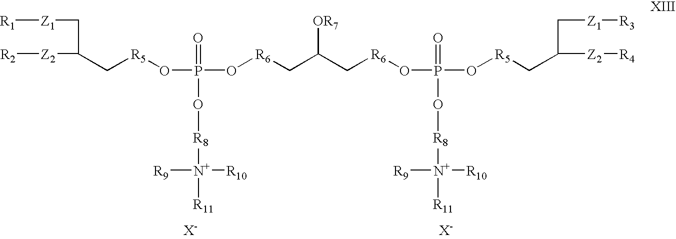
wherein Z₁ and Z₂ are the same or different and are —O—C(O)—, —O—, —S—, or —NH—C(O)—;
R₁, R₂, R₃, and R₄ are the same or different and are H, C₁ to C₃₂ saturated or unsaturated alkyl, alkenyl, or alkynyl, groups, optionally hydroxylated, aminenated, thiolated, epoxylated, cyclolated, PEGylated, halogenated, or substituted with combinations thereof;
R₅ and R₆ are the same or different and can be either absent or comprise a linker comprising a C₁-C₃₂ alkyl, substituted alkyl, cycloalkyl, substituted cycloalky, or an alkyloxy or substituted alkyloxy group such as PEGylated ether containing from 1 to 500 PEG (polyethylene glycol) units;
R₇, R₉, R₁₀ and R₁₁, are the same or different and are hydrogen, alkyl, substituted alkyl, alkyloxy, substituted alkyloxy, cycloalkyl, substituted cycloalkyl, alkenyl, alkynyl, alkanoyl, alkenoyl, alkynoyl, optionally hydroxylated, aminenated, thiolated, epoxylated, cyclolated, PEGylated, halogenated, or substituted with combinations thereof; or an alkyloxy or substituted alkyloxy group such as PEGylated ether containing from 1 to 500 PEG (polyethylene glycol) units; an amino acid, a peptide, a peptidomimetic moiety, a dipeptide, a polypeptide, folic acid, a protein, a carbohydrate, a saccharide or polysaccharide, a polyamine, a heterocyclic, a nucleoside, or a polynucleotide;
R₈ is C₂-C₃₂ alkyl, substituted alkyl, alkenyl, substituted alkenyl, alkyloxy or substituted alkyloxy;
X is a non-toxic anion.

14. The analog of claim 13 having the structure of formula XIV: 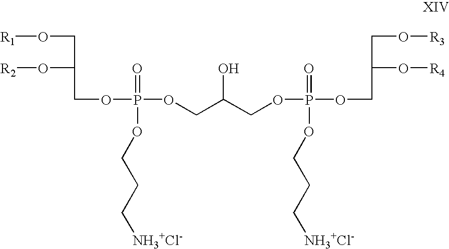
wherein R₁, R₂, R₃, and R₄ are the same or different and are H, C₁ to C₃₂ alkyl, alkenyl, or alkynyl.

15. The analog of claim 13 having the structure of formula XV: 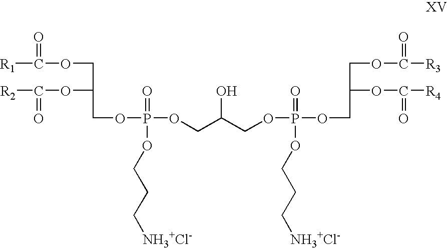
wherein R₁, R₂, R₃, and R₄ are the same or different and are H, or C₁ to C₃₁ alkyl, alkenyl, or alkynyl.

16. A lipid composition, comprising a cationic cardiolipin analog according to any of claims 1, 10 or 13.

17. The composition of claim 16, further comprising one or more co-lipids.

18. The composition of claims 17, wherein the co-lipids comprise neutral lipids.

19. The composition of claim 18, wherein the molar ratio of the cationic cardiolipin analog to neutral co-lipids is between about 9:1 to about 1:9.

20. The composition of claim 17, wherein the co-lipids are selected from a group consisting of phosphatidylethanolamine, phosphatidylcholine, sphingomyelin, sterol, tocopherol and cationic lipids.

21. The composition of claim 20, wherein the cationic lipid is selected from a group consisting of N-[1-(2,3-dioleyloxy)propyl]-N,N,N-trimethylammonium choloride (DOTMA) and 1,2,-dioleoyloxy-3-(trimethylammonium)-propane (DOTAP).

22. The composition of claim 20, wherein the phosphatidylcholine is selected from a group consisting of dimyristoylphosphatidyl choline, distearoylphosphatidylcholine, dioleylphosphatidyl choline, dipalmitoylphosphatidylcholine, diarachidonoylphosphatidylcholine, egg phosphatidylcholine, soy phosphatidylcholine, hydrogenated soy phosphatidylcholine, and mixtures thereof.

23. The composition of claim 20, wherein the sterol is selected from a group consisting of cholesterol, derivatives of cholesterol, coprostanol, cholestanol, cholestane, cholesterol hemisuccinate, cholesterol sulfate, and mixtures thereof.

24. The composition of claim 16, wherein the composition comprises liposomes.

25. The composition of claim 24, wherein the liposomes have a net neutral, negative or positive charge.

26. The composition of claim 24, wherein the liposomes are coated with biodegradeable polymers.

27. The composition of claim 24, wherein the liposomes comprise unilamellar vesicles, multilamellar vesicles or mixtures thereof.

28. The composition of claim 24, wherein the liposomes have a diameter of about 1 micron to 500 nm.

29. The composition of claim 16, further comprising a physiological acceptable vehicle.

30. The composition of claim 16, further comprising one or more active agents.

31. The composition of claim 30, wherein the active agent is selected from a group consisting of nucleoside analogue, nucleotide analogue, corticosteroid, non-steroidal anti-inflammatory agent, bioactive lipid, anticancer drugs, antibiotic, antifingal agent, oxidant, antiviral agent, nucleoside protein, peptide, polypeptide, polynucleotide, antisense polynucleotide, oligonucleotide and antisense oligonucleotide.

32. The composition of claim 16, further comprising a targeting agent.

33. The composition of claim 32, wherein the targeting agent is selected from a group consisting of a carbohydrate, protein and antibody.

34. A method of treating a disease, comprising administering the composition of claim 16 to a patient in need of treatment in an amount and at a location sufficient to treat the disease within the patient.

35. The method of claim 34, wherein the disease is cancer or a viral infection.

36. A method of introducing an active agent into a cell, comprising (a) preparing a composition comprising a cationic cardiolipin of claim 16 and said active agent (b) contacting said cell with said composition whereby said active agent is taken up into said cell.

37. The method of claim 36, wherein said contacting step occurs in vitro.

38. The method of claim 36, wherein said contacting step occurs in vivo.

39. A method of transfecting a cell with a polynucleotide, comprising (a) preparing a composition comprising a cationic cardiolipin analog of claim 16 and said polynucleotide (b) contacting said cell with said composition whereby said polynucleotide is taken up into said cell.

40. The method of claim 39, wherein the cells are in vitro.

41. The method of claim 39, wherein the cells are in vivo.

42. A kit for transfecting cells, said kit comprising a cationic cardiolipin of claim 16 and one or more elements selected from the group consisting of a polynucleotide, instructions for formulating the polynucleotide and the cationic cardiolipin into a preparation, instructions for transfecting cells using the cationic cardiolipin, reagents for facilitating transfection, containers for storing the cationic cardiolipin, containers for storing the polynucleotide, containers for storing the reagents, containers for storing a preparation including the cationic cardiolipin and polynucleotide, or containers for preparing the preparation, and materials to facilitate transfection.


SEQUENCE LISTING (1 sequence)

SEQ ID NO: 1 - DNA, 15 bp, Artificial Sequence
  Antisense polynucleotide targeting c-Raf
gtgctccattgatgc